(19) United States
(12) Patent Application Publication
(10) Pub. No.: US 2022/0395255 A1
(43) Pub. Date: Dec. 15, 2022

(54) TRANSDUCER FOR ULTRASOUND MEASURING SYSTEMS AND METHODS

(71) Applicant: Provisio Medical, Inc., San Diego, CA (US)

(72) Inventors: Stephen Eric Ryan, San Diego, CA (US); Nestor Cabrera-Munoz, San Diego, CA (US)

(21) Appl. No.: 17/804,518

(22) Filed: May 27, 2022

Related U.S. Application Data
(60) Provisional application No. 63/195,654, filed on Jun. 1, 2021.
(60) Provisional application No. 63/227,540, filed on Jul. 30, 2021.

(51) Int. Cl. A61B 8/00; A61B 8/08; B06B 1/06

(52) U.S. Cl. CPC A61B 8/4494; A61B 8/445; A61B 8/0891; B06B 1/0625; B06B 1/067; B06B 1/0685; B06B 2201/76

(57) ABSTRACT

An ultrasound transducer assembly that includes a piezoelectric layer configured to resonate and generate ultrasound signals around a predetermined ultrasound frequency in which the piezoelectric layer has a width to thickness ratio of at least about 0.6. A conductive matching layer is connected to the top surface of the piezoelectric layer to condition the ultrasound transducer for broad frequency bandwidth operation. A conductive backing layer is connected to the bottom surface of the piezoelectric layer. The ultrasound transducer assembly further includes a rigid body over which the conductive backing layer is positioned, the rigid body assembled for encompassing a central longitudinal axis of a catheter body. A signal and ground electrode may form a metallic layer over the top of or below each of the piezoelectric layers. Electrical waveguides may be connected to corresponding signal and ground electrodes of the transducers.

### REFERENCE TO RELATED APPLICATIONS

This application claims the benefit of U.S. Provisional Application Nos. 63/195,654, filed Jun. 1, 2021, and 63/227,540, filed Jul. 30, 2021, which are each hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of the Related Art

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Recent studies have shown that outcomes are significantly improved through the use of more advanced, more accurate imaging techniques.

### SUMMARY OF THE INVENTION

Embodiments of the present disclosure include novel implementations of ultrasound transducers and transducer arrays that can be used with imaging probes to approximate the dimensions and shapes of fluid-filled structures, including small-sized structures such as blood vessels. Some embodiments include transducers with components adapted to respond (i.e., echo) to the presence of particular materials (e.g., vessel walls, blood) without the complexity and footprint required by many common medical-grade imaging transducers such as in linear and linear-phased array arrangements. Some embodiments include transducers with components adapted to respond to the presence of particular materials without needing to provide signals representing enhanced detail/resolution/gradations of the materials provided by many common medical-grade imaging transducers. In some embodiments, a transducer of a transducer array includes reduced or omitted backing and/or matching layers when integrated into an imaging probe. After placement of multiple transducers, additional layers are placed over the transducers including, for example, an electrode layer and sealing layer. These layers may be adapted to gradually transition acoustic impedance between a piezoelectric layer of the transducer and the surface of the imaging probe so as to reduce impedance mismatch, enabling the transfer of acoustic energy into the imaged medium (e.g., vessel walls, blood), and reducing noise/signal distortion.

In some embodiments, a transducer assembly includes a piezoelectric layer configured to resonate and generate ultrasound signals around a predetermined ultrasound frequency and has a width to thickness ratio of at least about 0.6. A relatively thin backing layer, with a thickness that does not exceed the thickness of the piezoelectric layer, may be connected to the piezoelectric layer in order to attenuate signals directed internally from the face of the transducer and/or direct signals out from the face of the transducer. The backing layer may operate as an electrode by being constructed out of a conductive material (e.g., conductive epoxies). In some embodiments, the damping/backing layer is configured to provide about −20 dB or less of round-trip attenuation and/or may be substantially omitted. In some embodiments, the damping/backing layer is comprised substantially of a material having a Shore hardness of at least about 75 D. In some embodiments, a rigid body is connected to and positioned below the transducer and configured to attenuate ultrasound signals directed toward a central longitudinal axis of a catheter body in which it may be integrated and to direct ultrasound signals away from the central longitudinal axis of the catheter body. In some embodiments, the rigid body is comprised substantially of a material having a Shore hardness of at least about 65 D and may be configured to support and manage signals for multiple transducers (e.g., arranged/integrated circumferentially about a catheter) simultaneously.

In some embodiments, the width to thickness ratio of the piezoelectric layer is about 1 or more or about 3 or more. Higher ratios may increase the relative amplitude of signals but may detrimentally impact the uniformity (of frequency) of the signal. The rigid bodies such as disclosed herein may be arranged and configured to manage increased noise (e.g., ringing/reverberation) from such transducers without requiring corresponding increases in backing and/or matching layers.

In some embodiments, the transducer includes a matching layer having a thickness of less than about a quarter of the resonant wavelength of the matching layer material or substantially omits a matching layer. In some embodiments, the matching layer is comprised substantially of a material having a Shore hardness lower than that of the backing layer and/or rigid body.

In some embodiments, the transducer assembly includes a metallic conductive layer over a top side of the piezoelectric layer in which the metallic conductive layer is configured to operate as a signal or ground layer. The metallic conductive layer may be configured to match the acoustic impedance between the piezoelectric layer and media/structures external to the transducer and to offset the impact of a reduced or substantially omitted matching layer. In some embodiments, the metallic conductive layer is made substantially of a conductive epoxy and a malleable metal is applied (e.g., sputtered) on it to enhance adherence of an electrode connector (e.g., ribbon, wire, wedge, ball connector) to carry a voltage generated current from the piezoelectric layer to a signal generating/processing device. In some embodiments, the conductive epoxy has a thickness of less than about a quarter of a wavelength of a resonant frequency of the conductive epoxy material. In some embodiments, the malleable metal has a thickness of at least about 0.5 microns or of about 1 or more microns and can include chrome, gold, platinum, and/or copper. In some embodiments, the metallic conductive layer over the top side of the piezoelectric layer of each of the transducers is substantially only a malleable metal.

In some embodiments, an outermost protective layer is placed around the piezoelectric layer (and any intervening backing/matching/electrode layers and/or other components) and is configured to transition the acoustic impedance between the piezoelectric layer and an external environment outside of the transducer. In some embodiments, the outermost protective layer can include biocompatible polymers (e.g., parylenes) and/or medical grade adhesives and/or epoxies. In some embodiments, a lateral protective layer is positioned about lateral side surfaces of the piezoelectric layer and configured to suppress lateral vibrations, assisting in directing ultrasound signals from the piezoelectric layer away from the central longitudinal axis of a catheter/probe body in which it may be integrated. In some embodiments, the lateral protective layer can include medical grade adhesives and/or epoxies.

In some embodiments, the transducers are positioned onto a segment of an imaging probe that is hardened/reinforced in order to reduce distribution of vibrations from the transducers to the rest of the probe. This way, ultrasonic signals are substantially more directed toward targeted structures and cause less returned signal noise (e.g., ringing/reverberation) from the probe itself. The segment may include a ring-shaped structure selectively positioned beneath circumferential arrays of transducers. The ring may be constructed of metal or a hard polymer, for example, while remaining segments of a probe body are less rigid.

In some embodiments, transducers are positioned within cavities of a probe and may be form-fitting to the shape of the transducers. After placement of the transducers, layers of additional material may be applied to the external face of the transducers and the flexible body. These materials may include ground electrode material and/or sealing material, for example. A ground electrode layer common to multiples of the plurality of transducers may be applied and thereafter a sealing layer applied over the electrode layer and plurality of transducers. In some embodiments, the materials are designed to gradually transition the acoustic impedances between the piezoelectric layers and types of media expected to be present when the transducers are activated.

In some embodiments, a ultrasound transducer assembly includes a piezoelectric layer configured to resonate and generate ultrasound signals around a predetermined ultrasound frequency, wherein the piezoelectric layer has a width to thickness ratio of at least about 0.6; a conductive backing layer connected to the piezoelectric layer; and a rigid body over which the conductive backing layer is positioned, the rigid body assembled for encompassing a central longitudinal axis of a catheter body, where a rigidity of the rigid body is configured to attenuate ultrasound signals directed toward the central longitudinal axis of the catheter body and to direct ultrasound signals away from the central longitudinal axis of the catheter body.

In some embodiments, the width to thickness ratio of the piezoelectric layer is at least about 1. In some embodiments, the width to thickness ratio of the piezoelectric layer is between about 2 and 7. In some embodiments, the width of the piezoelectric layer is at least about a wavelength of a resonant frequency of an external environment outside of the catheter body. In some embodiments, the thickness of the piezoelectric layer is equal to or less than about one half a wavelength of a resonant frequency of the piezoelectric layer material. In some embodiments, the width to thickness ratio of the piezoelectric layer is less than about 20. In some embodiments, the width to thickness ratio of the piezoelectric layer is between about 3 and 15.

In some embodiments, the rigid body substantially includes a material having a Shore hardness of at least about 65 D. In some embodiments, the conductive backing layer is configured to provide about −20 dB or less of round-trip attenuation. In some embodiments, the conductive backing layer substantially includes a material having a Shore hardness of at least about 70 D. In some embodiments, the conductive backing layer has a thickness of about a tenth to about a half of the thickness of the piezoelectric layer. In some embodiments, the conductive backing layer is substantially omitted. In some embodiments, the rigid body includes a solid inner ring or polygon assembled for encompassing the central longitudinal axis of the catheter body. In some embodiments, the rigid body is configured and arranged to support multiple transducers.

In some embodiments, the ultrasound transducer assembly further includes a metallic conductive layer over a top side of the piezoelectric layer, the metallic conductive layer configured to operate as a signal or ground layer. In some embodiments, the metallic conductive layer substantially includes a conductive epoxy onto which a malleable metal is applied, and where the malleable metal has a thickness of at least about 0.5 microns. In some embodiments, a ribbon, ball, wedge, or wire electrode connector is attached to the conductive backing layer in order to carry a current between the piezoelectric layer and an external electrode. In some embodiments, a ribbon, ball, wedge, or wire electrode connector is attached to the metallic conductive layer over a top side of the piezoelectric layer in order to carry a current between the piezoelectric layer and an external electrode. In some embodiments, the malleable metal includes at least one of chrome, gold, platinum, or copper. In some embodiments, the metallic conductive layer substantially includes of a conductive epoxy onto which a malleable metal is applied and wherein the malleable metal has a thickness of at least about one micron. In some embodiments, the metallic conductive layer is configured to transition acoustic impedances between the piezoelectric layer and an external environment outside of the catheter body.

In some embodiments, the ultrasound transducer assembly includes an outermost protective layer over the piezoelectric layer and configured to transition an acoustic impedance between the piezoelectric layer and an external environment outside of the catheter body. In some embodiments, the ultrasound transducer assembly includes a lateral protective layer about lateral side surfaces of the piezoelectric layer, the lateral protective layer configured to suppress lateral-mode vibrations and to direct the ultrasound signals from the piezoelectric layer away from the central longitudinal axis of the catheter body. In some embodiments, the lateral protective layer has a hardness of greater than about 20 D Shore hardness. In some embodiments, the lateral protective layer has a hardness of between about 20 D and 90 D Shore hardness. In some embodiments, the lateral protective layer includes a non-conductive epoxy. In some embodiments, the tensile modulus of the lateral protective layer is between about 20 to 2,500 N/mm\hat{ }2. In some embodiments, the lateral protective layer has a width of about a tenth to about a third of the thickness of the piezoelectric layer. In some embodiments, the lateral protective layer is omitted. In some embodiments, the rigid body substantially includes metal. In some embodiments, the rigid body is manufactured to be radiopaque. In some embodiments, the metallic conductive layer includes a matching layer having a thickness of less than about a quarter of a wavelength of a resonant frequency in a matching layer material. In some embodiments, the matching layer is omitted. In some embodiments, the piezoelectric layer includes a single element. In some embodiments, the single element includes a crystal material or a ceramic material. In some embodiments, the single element includes a 2-2 or 1-3 composite configuration. In some embodiments, the single element 2-2 or 1-3 composite configuration includes a crystal or ceramic and a non-conductive epoxy. In some embodiments, the single element 2-2 or 1-3 composite configuration has a volume fraction of ceramic or crystal to non-conductive epoxy in the range of about 0.5 to 0.8.

In some embodiments, a transducer for ultrasound measuring includes a piezoelectric layer configured to resonate around a predetermined ultrasound wavelength and frequency; and a conductive backing layer directly connected to the bottom side of the piezoelectric layer, the conductive backing layer having a thickness that produces about −20 dB or less of round-trip attenuation, where the conductive backing layer is configured to operate as a conductive electrode of the transducer.

In some embodiments, the transducer further includes a rigid body over which the conductive backing layer is positioned, the rigid body assembled for encompassing a central longitudinal axis of an acoustic probe body, wherein the rigidity of the rigid body is configured to attenuate ultrasound signals directed toward the central longitudinal axis of the acoustic probe body and to direct ultrasound signals away from the central longitudinal axis of the acoustic probe body. In some embodiments, a width to thickness ratio of the piezoelectric layer is about 0.6 or greater. In some embodiments, a width to thickness ratio of the piezoelectric layer is between about 2 and 7.

In some embodiments, the transducer further includes a matching layer positioned over a top side of the piezoelectric layer, where the matching layer has a thickness of less than about a quarter of a resonant wavelength of a material of the matching layer. In some embodiments, the matching layer includes a metallic conductive material for operating as a signal or ground electrode of the transducer. In some embodiments, the metallic conductive layer substantially includes a conductive epoxy onto which a malleable metal is applied, and wherein the malleable metal has a thickness of at least about 0.5 microns. In some embodiments, a ribbon, ball, wedge, or wire electrode connector is attached to the conductive backing layer in order to carry a current between the piezoelectric layer and an external electrode. In some embodiments, a ribbon, ball, wedge, or wire electrode connector is attached to the metallic conductive material of the matching layer in order to carry a current between the piezoelectric layer and an external electrode.

In some embodiments, an ultrasound probe assembly includes a plurality of transducers integrated with a probe body, each of the plurality of transducers including a piezoelectric layer configured to resonate around a predetermined ultrasound wavelength and frequency; a signal and ground electrode, wherein the ground electrodes of each of the transducers include a common metallic layer formed over a top of each of the piezoelectric layers of the plurality of transducers; and a plurality of electrical waveguides extending from a proximal end of the probe body to the plurality of transducers and connected to corresponding signal and ground electrodes of the plurality of transducers.

In some embodiments, the common metallic layer includes at least one of chrome, gold, platinum, or copper. In some embodiments, the ultrasound probe assembly includes an electrode positioned below the piezoelectric layer of each of the plurality of transducers, the electrode configured to damp ultrasound waves generated by the piezoelectric layer. In some embodiments, the plurality of electrical waveguides includes micro-coaxial cables. In some embodiments, the plurality of electrical waveguides includes at least one elongated flexible printed circuit. In some embodiments, the piezoelectric layer has a width to thickness ratio of at least about 0.6. In some embodiments, the piezoelectric layer has a width to thickness ratio of at least about 1. In some embodiments, the piezoelectric layer has a width to thickness ratio of between about 2 and 7. In some embodiments, the width of the piezoelectric layer is at least about a wavelength of a resonant frequency of an external environment outside of the ultrasound probe assembly. In some embodiments, the thickness of the piezoelectric layer is equal to or less than about one half a wavelength of a resonant frequency of the piezoelectric layer material. In some embodiments, the width to thickness ratio of the piezoelectric layer is less than about 20. In some embodiments, the width to thickness ratio of the piezoelectric layer is between about 3 and 15.

In some embodiments, the plurality of transducers is positioned on a rigid body segment encompassing a central longitudinal axis of the ultrasound measuring probe assembly, wherein a rigidity of the rigid body is configured to attenuate ultrasound signals from each of the plurality of transducers directed toward the central longitudinal axis of the ultrasound measuring probe assembly and to direct ultrasound signals from each of the plurality of transducers away from the central longitudinal axis of the ultrasound measuring probe assembly. In some embodiments, the rigid body segment includes a material having a Shore hardness of at least about 65 D. In some embodiments, the rigid body segment forms at least one of a ring or polygon about which the plurality of transducers are annularly distributed. In some embodiments, the probe body includes micro-formed features into which the plurality of transducers is integrated. In some embodiments, each of the plurality of transducers is independently integrated with a respective feature of the probe body. In some embodiments, the respective feature includes a form-fitting cavity of the probe body arranged for supporting a respective transducer of the plurality of transducers and separating each respective transducer by at least about twice a wavelength of a resonant frequency of the transducer in an external environment outside of the ultrasound probe assembly.

In some embodiments, an ultrasound probe assembly includes a plurality of transducers integrated with a probe body, each of the plurality of transducers including a piezoelectric layer configured to resonate around a predetermined ultrasound wavelength and frequency, wherein the piezoelectric layer has a width to thickness ratio of at least about 0.6; and a plurality of electrical waveguides extending from a proximal end of the probe body to the plurality of transducers and connected to respective electrodes of the plurality of transducers.

In some embodiments, the piezoelectric layer has a width to thickness ratio of at least about 1. In some embodiments, the piezoelectric layer has a width to thickness ratio of between about 2 and 7. In some embodiments, the width of the piezoelectric layer is at least about a wavelength of a resonant frequency of an external environment outside of the ultrasound probe assembly. In some embodiments, the thickness of the piezoelectric layer is equal to or less than about one half a wavelength of a resonant frequency of the piezoelectric layer material. In some embodiments, the width to thickness ratio of the piezoelectric layer is less than about 20. In some embodiments, to thickness ratio of the piezoelectric layer is between about 3 and 15. In some embodiments, each of the plurality of transducers includes a metallic conductive layer over a top side of their respective piezoelectric layer, the metallic conductive layer configured to operate as a signal or ground electrode.

According to some embodiments, for each of the plurality of transducers, the metallic conductive layer substantially includes a conductive epoxy onto which a malleable metal is applied, wherein the malleable metal has a thickness of at least about 0.5 microns; an electrode connector is attached to the metallic conductive layer; and the transducer is positioned on a flexible circuit, the flexible circuit configured to carry a current to the plurality of electrical waveguides from the electrode connector. In some embodiments, the electrode connector includes at least one of ribbon, ball, wedge, or wire electrode connector. In some embodiments, the malleable metal includes at least one of chrome, gold, platinum, or copper.

In some embodiments, each of the plurality of transducers is positioned on a common rigid body segment encompassing a central longitudinal axis of the ultrasound probe assembly, wherein a rigidity of the common rigid body segment is configured to attenuate ultrasound signals from each of the plurality of transducers directed toward the central longitudinal axis of the ultrasound probe assembly and to direct ultrasound signals from each of the plurality of transducers away from the central longitudinal axis of the ultrasound probe assembly. In some embodiments, the rigid body segment substantially includes a material having a Shore hardness of at least about 65 D. In some embodiments, the plurality of electrical waveguides includes micro-coaxial cables. In some embodiments, the plurality of electrical waveguides includes at least one elongated flexible printed circuit.

In some embodiments, the ultrasound measuring probe assembly includes a plurality of transducers integrated with a probe body, each of the plurality of transducers including a piezoelectric layer configured to resonate around a predetermined ultrasound wavelength and frequency and wherein the plurality of transducers has a center-to-center pitch of at least about twice the wavelength of the transducers' resonant frequency in an external environment outside of the ultrasound measuring probe assembly; and a plurality of electrical waveguides extending from a proximal end of the probe body to the plurality of transducers and connected to respective electrodes of the plurality of transducers.

In some embodiments, the plurality of transducers has a center-to-center pitch of at least about 0.1 mm. In some embodiments, the plurality of transducers has a center-to-center pitch of at least about 0.5 mm. In some embodiments, the piezoelectric layer of each of the plurality of transducers has a width to thickness ratio of at least about 0.6. In some embodiments, the piezoelectric layer of each of the plurality of transducers has a width to thickness ratio of at least about 1. In some embodiments, the piezoelectric layer of each of the plurality of transducers has a width to thickness ratio of between about 2 and 7. In some embodiments, the width of the piezoelectric layer is at least about a wavelength of a resonant frequency of an external environment outside of the ultrasound measuring probe assembly. In some embodiments, the thickness of the piezoelectric layer is equal to or less than about one half a wavelength of a resonant frequency of the piezoelectric layer material. In some embodiments, the width to thickness ratio of the piezoelectric layer is less than about 20. In some embodiments, to thickness ratio of the piezoelectric layer is between about 3 and 15. In some embodiments, each of the plurality of transducers includes a metallic conductive layer over a top side of their respective piezoelectric layer, the metallic conductive layer configured to operate as a signal or ground electrode.

In some embodiments, for each of the plurality of transducers, the metallic conductive layer substantially includes a conductive epoxy onto which a malleable metal is applied, where the conductive epoxy has a thickness of less than about a quarter of a wavelength of a resonant frequency of the conductive epoxy material, and where the malleable metal has a thickness of at least about 0.5 microns; an electrode connector is attached to the metallic conductive layer; and the transducer is positioned on a flexible circuit, the flexible circuit configured to carry a current to the plurality of electrical waveguides from the electrode connector. In some embodiments, the electrode connector includes at least one of ribbon, ball, wedge, or wire electrode connector. In some embodiments, the malleable metal includes at least one of chrome, gold, platinum, or copper.

In some embodiments, each of the plurality of transducers is positioned on a rigid body assembled for encompassing a central longitudinal axis of the ultrasound measuring probe assembly, wherein the rigidity of the rigid body is configured to attenuate ultrasound signals from each of the plurality of transducers directed toward a central longitudinal axis of the ultrasound measuring probe assembly and to direct ultrasound signals from each of the plurality of transducers away from the central longitudinal axis of the ultrasound measuring probe assembly. In some embodiments, the rigid body segment substantially includes a material having a Shore hardness of at least about 65 D. In some embodiments, the plurality of electrical waveguides includes micro-coaxial cables. In some embodiments, the plurality of electrical waveguides includes at least one elongated flexible printed circuit.

According to some embodiments, a method of integrating an array of ultrasound transducers into a measuring probe assembly is disclosed herein, the method including assembling a plurality of ultrasound transducers configured to resonate around a predetermined ultrasound wavelength and frequency; positioning each transducer of the plurality of transducers on or within micro-formed features of a body of the measuring probe assembly; attaching an electrode to the top side of a piezoelectric layer of each transducer, the electrode configured and arranged to provide a transition in acoustic impedance between the piezoelectric layer and an environment outside of the plurality of ultrasound transducers; and connecting a plurality of electrical waveguides between the plurality of ultrasound transducers and a proximal end of the measuring probe assembly.

According to some embodiments, attaching the electrode includes forming a common layer of conductive material over the plurality of ultrasound transducers after the positioning of each transducer on or within the micro-formed features. In some embodiments, the micro-formed features include form-fitting cavities into which respective ones of the plurality of transducers are positioned. In some embodiments, positioning each transducer includes positioning each transducer on a rigid body segment, the rigid body segment encompassing a central longitudinal axis of the measuring probe assembly, wherein a rigidity of the rigid body segment is configured to attenuate ultrasound signals from each of the plurality of transducers directed toward the central longitudinal axis of the measuring probe assembly and to direct ultrasound signals from each of the plurality of transducers away from the central longitudinal axis of the measuring probe assembly. In some embodiments, the rigid body segment substantially includes a material having a Shore hardness of at least about 65 D. In some embodiments, the piezoelectric layer of each of the plurality of transducers has a width to thickness ratio of at least about 0.6. In some embodiments, the piezoelectric layer of each of the plurality of transducers has a width to thickness ratio of at least about 1. In some embodiments, the piezoelectric layer of each of the plurality of transducers has a width to thickness ratio of between about 2 and 7. In some embodiments, the width of the piezoelectric layer is at least about a wavelength of a resonant frequency of an external environment outside of the ultrasound measuring probe assembly. In some embodiments, the thickness of the piezoelectric layer is equal to or less than about one half a wavelength of a resonant frequency of the piezoelectric layer material. In some embodiments, the width to thickness ratio of the piezoelectric layer is less than about 20. In some embodiments, the width to thickness ratio of the piezoelectric layer is between about 3 and 15. In some embodiments, positioning the transducers includes positioning the transducers apart by at least about twice a wavelength of a resonant frequency of the transducer in an external environment outside of the ultrasound measuring probe assembly.

In some embodiments, the method may further include positioning each of the plurality of transducers on a flexible circuit before positioning the plurality of transducers on or within the micro-formed features of the body of the measuring probe assembly; forming a metallic conductive layer over a top side of the piezoelectric layer of each of the transducers, the metallic conductive layer substantially including a conductive epoxy having a thickness of less than about a quarter of a wavelength of a resonant frequency of the conductive epoxy material; applying a malleable metal on the metallic conductive layer, wherein the malleable metal has a thickness of at least about 0.5 microns; attaching an electrode connector between the metallic conductive layer and the flexible circuit; and connecting the flexible circuit to the plurality of electrical waveguides. In some embodiments, the electrode connector includes at least one of ribbon, ball, wedge, or wire electrode connector. In some embodiments, the malleable metal includes at least one of chrome, gold, platinum, or copper. In some embodiments, the ultrasound transducers are substantially without a backing layer when they are positioned on or within the micro-formed features of the body of the measuring probe assembly. In some embodiments, the metallic conductive layer over a top side of the piezoelectric layer of each of the transducers substantially includes only a malleable metal with a thickness of at least about 0.5 microns. In some embodiments, the micro-formed features are at least one of micro-molded, micro-extruded, or micro-machined. In some embodiments, the plurality of electrical waveguides includes micro-coaxial cables. In some embodiments, the plurality of electrical waveguides includes at least one elongated flexible printed circuit.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system with multiple transducers according to some embodiments.

FIG. 2A is an illustrative side perspective diagram of an ultrasound catheter probe placed within a lumen according to some embodiments.

FIG. 2B is a cross-sectional perspective diagram of the ultrasound catheter probe of FIG. 2A.

FIG. 2C is another cross-sectional perspective diagram of the ultrasound catheter probe of FIG. 2A.

FIG. 3A is an illustrative diagram of an imaging probe assembly for integrating an ultrasound transducer array according to some embodiments.

FIG. 3B is an illustrative drawing of a holder cap for the assembly of FIG. 3A according to some embodiments.

FIG. 3C is an illustrative cross-sectional diagram of the imaging probe assembly of FIG. 3A integrated with an array of ultrasound transducers according to some embodiments.

FIG. 3D is an illustrative perspective view of the imaging probe assembly of FIG. 3A integrated with an array of ultrasound transducers according to some embodiments.

FIG. 4A is an illustrative diagram of a transducer according to some embodiments.

FIG. 4B is an illustrative diagram of a transducer according to some embodiments.

FIG. 4C is an illustrative diagram of a transducer according to some embodiments.

FIG. 5A is an illustrative chart of a pulse (“chirp”) for activating a transducer according to some embodiments.

FIG. 5B is an illustrative chart of the frequency spectrum of a pulse for activating a transducer according to some embodiments.

FIG. 6A is an illustrative diagram of a piezoelectric transducer for integration into an imaging probe transducer array according to some embodiments.

FIG. 6B is an illustrative chart of a response over time to a pulse of determined frequency in the transducer of FIG. 6A.

FIG. 7A is an illustrative diagram of a piezoelectric transducer for integration into an imaging probe transducer array according to some embodiments.

FIG. 7B is an illustrative chart of a response over time to a pulse of determined frequency in the transducer of FIG. 7A with a reduced or omitted backing layer according to some embodiments.

FIG. 8A is an illustrative cross-sectional diagram of a transducer positioned on a probe body segment according to some embodiments.

FIG. 8B is an illustrative cross-sectional diagram of a transducer positioned on a hardened probe body segment according to some embodiments.

FIG. 9A is an illustrative diagram of an imaging probe inserted into a lumen according to some embodiments.

FIG. 9B is an illustrative diagram of an ultrasound transducer of FIG. 9A imaging a lumen wall.

FIG. 10A is an illustrative chart of a response over time to a pulse of a determined frequency in an ultrasound transducer having a backing layer.

FIG. 10B is an illustrative chart of a response over time to a pulse of a determined frequency in an ultrasound transducer without all or a portion of a backing and/or matching layer according to some embodiments.

FIG. 11 is an illustrative flowchart of a process for assembling an imaging probe according to some embodiments.

FIG. 12 is an illustrative flowchart of a process for processing signals from a transducer with a reduced or omitted backing and/or matching layer according to some embodiments.

FIG. 13 is an illustrative flowchart of a process for measuring a structure using an ultrasound transducer array according to some embodiments.

FIG. 14A is an illustrative diagram of a transducer array integrated with a probe body according to some embodiments.

FIG. 14B is an illustrative diagram of a probe body for the assembly of FIG. 14A according to some embodiments.

FIG. 14C is an illustrative cross-sectional diagram of the transducer array probe assembly of FIG. 14A according to some embodiments.

FIG. 15 is an illustrative flowchart of a process for assembling a transducer array imaging probe according to some embodiments.

FIG. 16 is an illustrative flowchart of a process for assembling a transducer array imaging probe according to some embodiments.

FIG. 17A is an illustrative diagram of an ultrasound probe integrated with a probe body according to some embodiments.

FIG. 17B is an illustrative cross-sectional diagram of the ultrasound probe of FIG. 17A according to some embodiments.

FIG. 18A is an illustrative diagram of a probe body according to some embodiments.

FIG. 18B is an illustrative diagram of a transducer array integrated with a probe body of FIG. 18A according to some embodiments.

FIG. 18C is an illustrative cross-sectional diagram of the transducer array probe assembly of FIG. 18B according to some embodiments.

FIG. 19 is an illustrative diagram of an angioplasty catheter integrated with an array of transducers according to some embodiments.

### DETAILED DESCRIPTION OF THE INVENTION

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure. It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and are typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure. Some systems are described in which multiple lumen wall distances are measured and a shape of the wall is calculated using the distance measurements such as described in U.S. Pat. No. 10,231,701 filed Mar. 14, 2014 (the '701 Patent), the entire contents of which are herein incorporated by reference.

While current IVUS and OCT systems provide additional and more detailed information compared to angiograms, these IVUS and OCT systems introduce significant additional time, cost and complexity into minimally invasive procedures. The components of these systems (e.g., transducers, wires, imaging circuitry, fiber-optics, etc.) can occupy a large footprint within the blood vessel and must often be deployed independently and at separate times from interventional procedures (e.g., angioplasty). In a typical IVUS system, for example, each ultrasound transducer of an array of transducers includes a piezoelectric layer with dimensions tailored to resonate at frequencies for detecting physiological properties (e.g., occlusion, calcification, etc.) of a blood vessel. Added to the piezoelectric layer is typically a matching layer providing the transducer with an acoustic impedance interface tailored (“matched”) to efficiently transmit the acoustic energy of ultrasound waves by gradually transitioning the acoustic impedances from the piezoelectric layer to tissue that is being imaged. A matching layer may also further be adapted to broaden the ultrasonic frequency range (bandwidth) that the transducer can measure. A backing layer is also added in order to prevent ultrasound waves from traveling in undesired inward direction (i.e., toward a central longitudinal axis of the catheter body) and generating signal noise from excessive ringing. However, these components can greatly increase the footprint of an IVUS system intended to fit within small areas such as blood vessels.

Further, the images produced by IVUS and OCT systems may not directly provide useful information about blood vessels and are typically subject to nonconforming interpretations of different physicians. Thus, there is a need for improved, reduced footprint, and more efficient imaging systems and components for obtaining information about a vessel or structure, particularly information about the diameter, area, and multi-dimensional profile of a vessel or structure, while not sacrificing speed and accuracy for timely, efficient, and effective treatment.

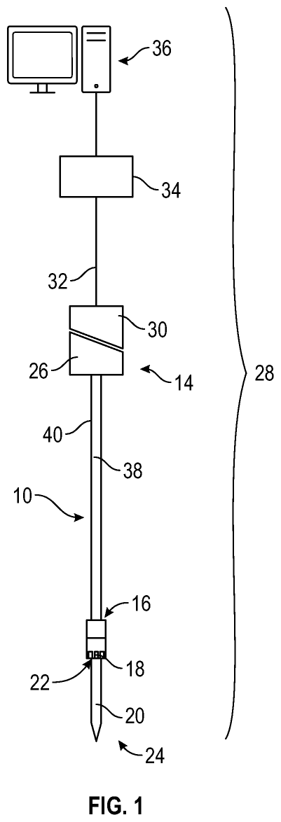

FIG. 1 is an illustrative diagram of an ultrasound catheter probe system 28 with multiple transducers according to some embodiments. In certain embodiments, an ultrasound imaging probe 10 includes a body member 40 having a proximal end 14 and a distal end 16. In certain embodiments, the probe 10 includes a plurality of transducers 18. In certain embodiments, the probe 10 comprises an elongated tip 20 having a proximal end 22 and a distal end 24. In certain embodiments, the probe 10 includes a proximal connector 26 which connects probe 10 to other components of system 28, for example, a data acquisition unit 34 and computer system 36. In certain embodiments, the medical device 10 is part of a system 28 that includes a distal connector 30, electrical conductor 32, the data acquisition unit 34, and the computer system 36.

In some embodiments, the body member 40 is tubular and has a central lumen 38 for containing various connectors and channels that extend toward the distal end 16. In some embodiments, the body member 40 has a diameter of about 1,500 μm, 650 μm, or less. These dimensions are illustrative and not intended to be limiting. In some embodiments, the diameter of the probe 10 will depend on the type of device that the probe 10 is integrated with and where the probe 10 will be used (e.g., in a blood vessel), which will become apparent to those of ordinary skill in the art in view of the present disclosure.

In certain embodiments, the proximal end 14 of the body member 40 is attached to the proximal connector 26. In some embodiments, the probe 10 includes an elongated tip 20 in which its proximal end 22 is attached to the distal end 16 of the body member 40. The elongated tip 20 may be constructed with an appropriate size, strength, and flexibility to be used for guiding the probe 10 through a body lumen (e.g., a blood vessel). In certain embodiments, the elongated tip 20 and/or other components of the probe 10 may include one or more radiopaque markers (e.g., visible to angiography) for precisely guiding the catheter through a lumen and positioning one or more transducers 18 in the desired location. In some embodiments, the probe 10 and the distal end 16 are constructed and arranged for rapid exchange use. In certain embodiments, the body member 40 and the elongated tip 20 may be made of resilient flexible biocompatible material such as is common for IVUS and intravascular catheters known to those of ordinary skill in the art.

In certain embodiments, the body member 40 has a tubular shape with a central lumen 38. In some embodiments, the probe 10 may have lumens for use with various features not shown (guidewires, fiberoptics, saline flush lumens, electrical connectors, etc.). In some embodiments, the outer diameter of the body member 40 and the elongated tip 20, if present, is substantially consistent along its length and does not exceed a predetermined amount.

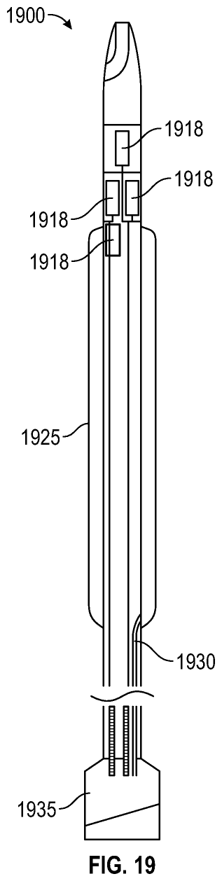

The one or more transducers 18 may be incorporated with the body member 40 of the distal end 16 such as described further herein such as to reduce the footprint of the body member 40. The one or more transducers 18 may be connected by one or more conductors extending through the lumen 38 to the data acquisition unit 34. In certain embodiments, signals received and processed by the data acquisition unit 34 are then processed by the computer system 36. In certain embodiments, the computer system 36 is programmed to store and analyze the signals (e.g., calculate distance measurements between the catheter and lumen wall). In some embodiments, by reducing the footprint of the body member 40, the space saved may be utilized to incorporate additional features (e.g., an expandable balloon and a balloon media lumen such as shown in FIG. 19). In some embodiments, the area of the face of each transducer's piezoelectric layer is at least about 2500 square microns and/or has a width of about 50 microns or more. These dimensions are illustrative and not intended to be limiting.

In some embodiments, the one or more transducers 18 are ultrasonic. In some embodiments, the one or more transducers 18 are piezoelectric. The one or more transducers 18 may be built using single element piezoelectric ceramic or crystal material, as well as piezoelectric composites of ceramic or crystal material with non-conductive epoxies. In some embodiments, the composites include a 2-2 or 1-3 configuration having a volume fraction of ceramic or crystal to non-conductive epoxy in the range of about 0.5 to 0.8. These values are illustrative and not intended to be limiting. In some embodiments, the one or more transducers 18 use piezoelectric crystals composed of Pb(Mg_{1/3}Nb_{2/3})O₃—PbTiO₃ (PMN-PT) or other types of piezoelectric materials with dimensions configured to resonate, for example, at predetermined frequencies. In some embodiments, the one or more transducers 18 are photoacoustic transducers and/or ultrasonic sensors that use MEMS (Microelectromechanical Systems) technology, such as but not limited to PMUTs (Piezoelectric Micromachined Ultrasonic Transducers) and CMUTs (Capacitive Micromachined Ultrasonic Transducers).

In some embodiments, the operating/center resonant frequency for the one or more transducers 18 may be in the range of about 8 to about 50 MHz or even up to about 60 MHz, depending on the dimensions and characteristics of the one or more transducers 18 and requirements of the particular application. Generally, higher frequency of operation provides better resolution and reduces the size of probe 10. However, the tradeoff for this higher resolution and smaller probe 10 size may be a reduced depth of penetration into the tissue of interest and increased echoes from the blood itself (making the image more difficult to interpret). Lower frequency of operation is more suitable for imaging in larger vessels or within structures such as the chambers of the heart. Although specific frequency ranges have been given, these ranges given are illustrative and not limiting. The one or more transducers 18 may produce and receive any frequency that leaves the one or more transducers 18, impinges on some structure or material of interest and is reflected back to and picked up by one or more transducers 18.

The operating/center resonant frequency and bandwidth of one or more transducers 18 is generally related to the thickness of transducer materials generating or responding to ultrasound signals. For example, in some embodiments, the one or more transducers 18 includes a piezoelectric material such as quartz and/or lead-zirconate-titanate (PZT). A thicker layer will generally respond to a longer wavelength and lower frequency and vice versa. For example, a 50-micron thick layer of PZT may have a resonant frequency of about 40 MHz, a 65-micron thick layer may have a resonant frequency of about 30 MHz, and a 100-micron thick layer may have a resonant frequency of about 20 MHz. As further described herein, matching and backing layers may be included, reduced, or omitted which affect the bandwidth and other characteristics of the one or more transducers 18.

In some embodiments, a resonant frequency of some of the one or more transducers 18 may be centered around 10, 15, 20, 25, or 30 MHz while other transducers of the one or more transducers 18 may have a resonant frequency centered around 35, 40, 45, or 50 MHz, for example. The respective materials and dimensions of the transducer layers may be configured accordingly. Subsets of the one or more transducers 18 may be activated at the same time while other subsets are activated at a separate time. In some embodiments, an electronic switch is utilized to switch connections between different transducers 18 or subsets of the one or more transducers 18.

In some embodiments, the probe 10 is connected with an actuating mechanism. In certain embodiments, the actuating mechanism rotates and/or longitudinally moves at least some portion of the probe 10 and its transducers 18. In certain embodiments, a controlled longitudinal and/or radial movement permits the probe 10 to obtain ultrasound readings from different perspectives within a surrounding structure, for example. Positioning the probe 10 and its transducers 18 in target locations may be augmented/guided by real-time imaging feedback provided by the transducers 18 and the system 28. In certain embodiments, relative positions of the probe 10 may be tracked and recorded during such processes (e.g., by using an encoder or other position sensing tool).

In some embodiments, the system 28 is programmed to analyze and identify characteristics of the medium (e.g., blood) between the probe 10 and structure in order to determine where the medium ends with respect to the structure (e.g., blood vessel wall). In some embodiments, multiple ultrasound images of the blood may be generated and the differences between the images are used to identify movement/change of the blood over time (e.g., as a result of a heart pumping). In some embodiments, doppler echo signals are used to determine these differences. Because the blood vessel wall does not have the same movement/change characteristics as the blood, the amount (or distance) between the probe 10 and blood vessel wall can be calculated. In some cases, reliance on the blood images without substantial reliance on images of the blood vessel wall may be used to determine the distance between the probe 10 and the blood vessel wall.

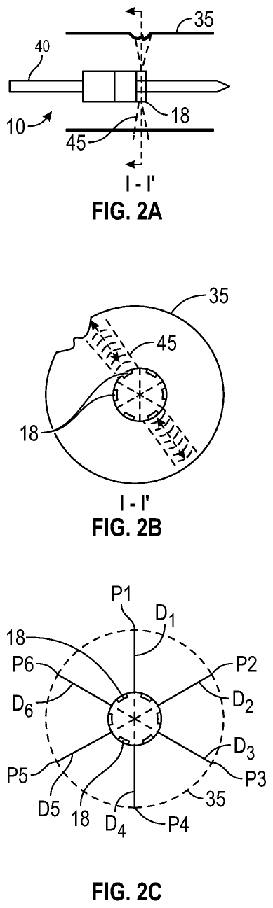

FIG. 2A is an illustrative side perspective diagram of an ultrasound catheter probe 10 placed within a lumen according to some embodiments. FIG. 2B is a cross-sectional perspective diagram of the ultrasound catheter probe 10 across lines I-I′ of FIG. 2A. FIG. 2C is another cross-sectional perspective diagram of the ultrasound catheter probe 10 across lines I-I′ of FIG. 2A. The probe 10 is shown inserted into a lumen 35. In certain embodiments, the computer system 36 is programmed to cause the one or more transducers 18 to generate pulses 45. In certain embodiments, each of the pulses 45 is incident on different portions of the lumen 35. The one or more transducers 18 generate electromagnetic signals respective to the pulses that reflect back (i.e., echoes) from the media and the lumen 35 adjacent to the probe 10. In certain embodiments, the electromagnetic signals are processed by a signal processor and the computer system 36.

In certain embodiments, the computer system 36 can be programmed to analyze and distinguish pertinent imaging data within the frequency response received by the one or more transducers 18. Because the one or more transducers 18 may be configured and arranged with a reduced footprint, including reduced and/or omitted backing and matching layers, the signals associated with imaging data may be obscured by additional noise associated with the activating pulse 45. In some embodiments, an envelope signal associated with the activating pulse 45 is detected and distinguished within the return signals to identify a transition between media and/or structural features. Based on the distinction, a distance measurement may be calculated between the transducer 18 of probe 10 and the transition location.

Other pulses may be similarly delivered/echoed using other transducers 18. In some embodiments, these pulses may be delivered simultaneously or at different times. In some embodiments, along with identifying and associating the signals with respective transducers 18, the computer system 36 can be programmed to analyze the signals and calculate a radial distance measurement (e.g., D1, D2, . . . , D6) between each transducer 18 and lumen 35. This may be done, for example, by utilizing time-of-flight information of the echo signals and previously determined/differentiated signatures representative of a lumen wall (e.g., of the lumen 35) and a particular medium (e.g., blood) between the transducer 18 and the lumen 35. Exemplary systems and methods for making such calculations are described, for example, in U.S. Pat. No. 10,231,701 filed Mar. 14, 2014 (the '701 patent), the entire contents of which are herein incorporated by reference.

In certain embodiments, based on distance calculations (D1, D2, . . . , D6), the shape and dimensions of the lumen 35 may be estimated by further utilizing information including one or more dimensions of the probe 10. In certain embodiments, the estimating may include applying interpolation and/or other mathematical fitting techniques. For example, in certain embodiments, the relative positions of points (p1, . . . , p6) about lumen 35 may first be calculated and a curve fitting algorithm (e.g., spline interpolation) is applied to generate a two-dimensional slice representation of the lumen 35. As described in the '701 patent, multiple slices can be calculated by taking sets of ultrasound readings along the longitudinal extent of the lumen 35 and combining them to generate a three-dimensional representation.

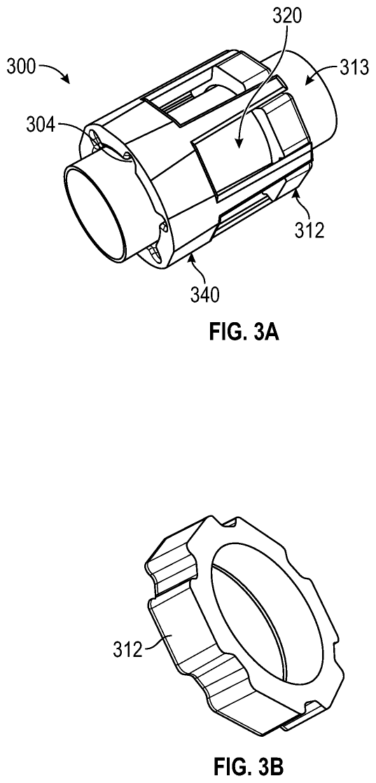

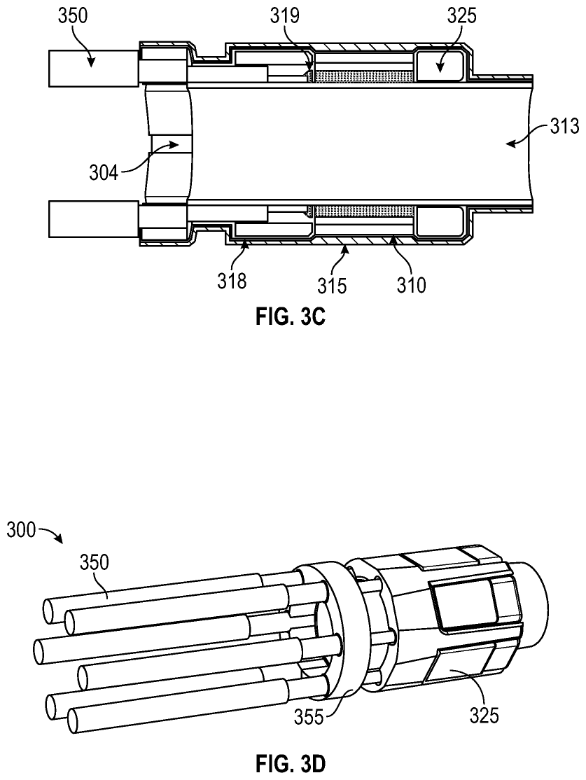

FIG. 3A is an illustrative diagram of an imaging probe assembly 300 for integrating an ultrasound transducer array according to some embodiments. FIG. 3B is an illustrative drawing of a holder cap 312 for the assembly of FIG. 3A. In certain embodiments, the assembly 300 includes a mounting body 313 around which a transducer holder body 340, the holder cap 312, and a cable holder 355 are positioned (e.g., by sliding and affixing them over the mounting body 313). In certain embodiments, the positioning of the holder body 340 and the holder cap 312 form one or more cavities 320 into which the one or more transducers 325 can be placed. The holder body 340 includes channels 304 for holding electrical cables/waveguides 350. In some embodiments, the electrical cables/waveguides 350 are micro-coaxial cables. In some embodiments, at least one of the electrical cable waveguides is an elongated flexible printed circuit.

In some embodiments, the cavities 320 (and transducers placed inside them) are separated (e.g., circumferentially, linearly) by a center-to-center pitch of at least about twice the wavelength of the ultrasound signals in the medium (e.g., blood) in which they are transmitted. In some embodiments, the center-to-center pitch is about at least 0.1 mm and, in some embodiments, of at least about 0.5 mm. The transducers 325 may be configured to operate independently (e.g., in contrast to linear or linear-phased array configurations) without being unduly interfered with from each other's signals.

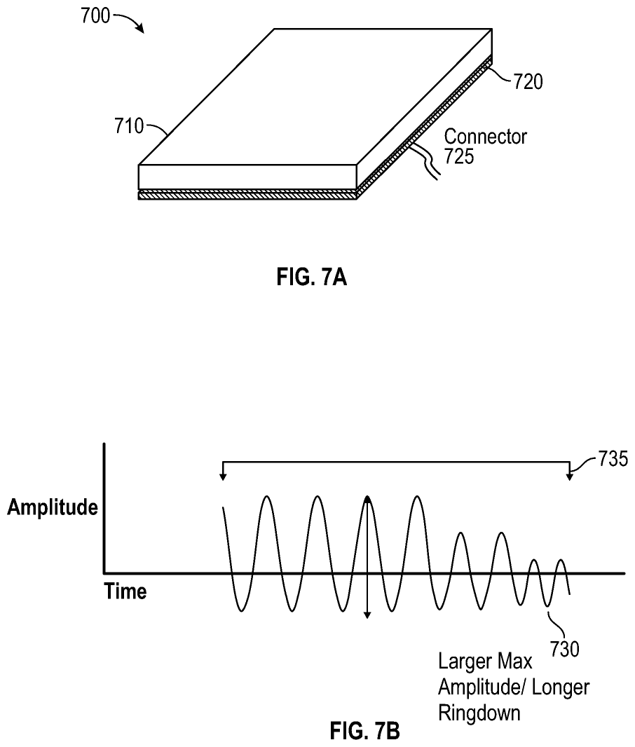

FIG. 3C is an illustrative cross-sectional diagram of an imaging probe assembly 300 integrated with an array of ultrasound transducers 325 according to some embodiments. The transducers 325 are placed in the cavities 320 to be in contact with electrical signal contacts such as a signal electrode 319. The transducers 325 can be in accordance with any of the transducers described herein, such as the transducer of FIG. 7A, for example, without backing, protective, and/or matching layers. The signal electrode 319 or signal electrode layer may be a component of the transducer 325 or inserted separately into the cavity 320. In some embodiments, the elements of the probe assembly 300 are micro-formed (e.g., micro-machined/extruded/molded). In some embodiments, the cavities 320 separate each transducer 325 by at least about twice a wavelength of a resonant frequency of the transducer 325 in an external environment outside of the ultrasound probe assembly 300.

In some embodiments, the mounting body 313 is a rigid or semi-rigid body segment composed and arranged to inhibit at least a portion of acoustic signals from emanating from the back or bottom sides of the transducers 325. The mounting body 313 may be constructed to be more rigid with respect to layers of material applied to the surfaces of the transducers 325 so that resistance to mechanical vibrations is relatively lower in the desired direction of acoustic signal travel. The rigidity and resistance to mechanical vibrations of the mounting body 313 may be based on a relatively larger and extensive structure with respect to each transducer 325. In some embodiments, the mounting body 313 includes a solid cylinder that may comprise metals or other hard/stiff materials with a Shore hardness of at least about 65 D.

In some embodiments, the mounting body 313 is manufactured/assembled to be of a hardened/rigid material and extent so as to attenuate ultrasound signals directed toward the central longitudinal axis of a catheter body (e.g., the body member 40 of FIG. 1) and to direct ultrasound signals away from the central longitudinal axis of the catheter body. For example, the mounting body 313 may comprise steel or a sufficiently hardened polymer (e.g., carbon fiber). In some embodiments, the diameter of a rigid body segment of the mounting body 313 is between about 500 μm and 2 mm. In some embodiments, the diameter of the mounting body 313 is about 1 mm. In some embodiments, the mounting body 313 is assembled to encompass the central longitudinal axis of a catheter body (e.g., the body member 40 of FIG. 1).

In some embodiments, a rigid body segment is positioned beneath the transducers 325. In some embodiments, the rigid body segment is a solid body that substantially incorporates a material having a Shore hardness of at least about 65 D. The rigid segment's extent may be limited to being directly positioned beneath the transducers 325 and may be formed as a solid ring in certain embodiments. Other adjacent segments of the probe/catheter may be selected to provide sufficient flexibility for the particular application and can be substantially less stiff than the rigid segment (e.g., a coronary catheter may require a high degree of flexibility). In some embodiments, the rigid body segment is configured to be radiopaque and permit an imaging device (e.g., radiography device) to locate its position within a structure.

In some embodiments, the holder body 340 represents a polygon. In some embodiments, each of the transducers 325 individually occupies an outer edge/side of the polygon. In some embodiments, the polygon has about 5 to 16 edges/sides and may have a corresponding transducer 325 on each edge/side. That way, the transducers 325 may be annularly distributed about the imaging probe assembly 300.

In certain embodiments, an adhesive/sealant 318 may be applied around the edges of the transducers 325 to secure the transducers 325 within the cavities 320 and prevent external materials from entering the sealed areas. The adhesive/sealant 318 may be an epoxy, acrylic, or ultraviolet-light curable acrylic, for example, and can further be applied around the transducers 325, the holder body 340, and/or the cap 312 in order to smooth/even the surfaces of the different components with respect to each other.

In certain embodiments, a ground/signal electrode 310 (or plurality of ground/signal electrodes) may then be placed over the transducers 325 for use in measuring charge across the transducers 325. The ground/signal electrode 310 may be a conductive layer sputtered or laminated over the surfaces of and common to each of the transducers 325, for example, as further described herein. In some embodiments, the ground/signal electrode 310 comprises metal including, for example, gold, copper, chrome, titanium, brass, silver, and/or platinum. In some embodiments, the ground/signal electrode 310 is comprised substantially of metal.

After application of the adhesive/sealant 318 and the electrode 310, an insulating coating 315 may be applied over the holder body 340 and the cap 312 to insulate the transducers 325 from environmental factors. In some embodiments, the materials covering the transducers 325, including the electrode 310 and the coating 315, are selected and applied to gradually transition the acoustic impedance between the resonant material of the transducers 325 (e.g., a piezoelectric crystal) and targeted structures that will be imaged by the transducers 325. In some embodiments, the coating 315 comprises polymer/parylene materials such as Parylene C.

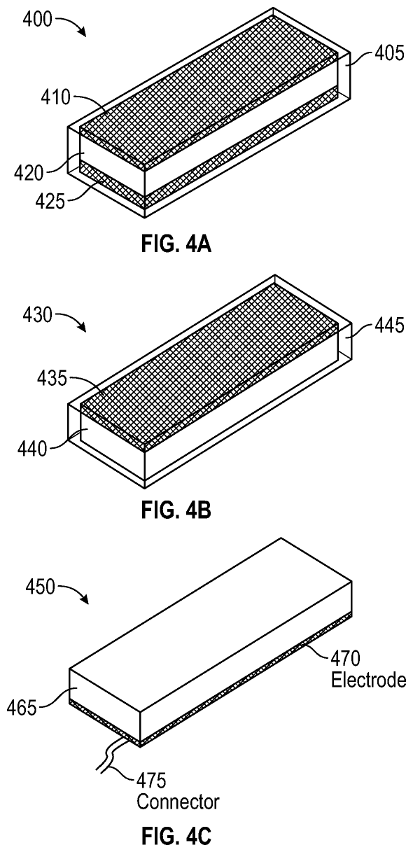

In some embodiments, the materials used for elements/layers below the piezoelectric components (e.g., under piezoelectric layers/crystals 420, 440, 465, 610, and 710 of FIGS. 4A-C, 6A, and 7A) of transducers 325 are constructed to be harder and more resistant to acoustic vibration than materials/layers placed over the piezoelectric components. That way, acoustic vibrations/signals generated from the piezoelectric components are directed more outwardly from transducers 325. In some embodiments, the Shore hardness of a matching/coating layer of the transducer 325 is about 55 D and a bonding layer attaching the transducers 325 to the body 313 is about 85, for example.

FIG. 4A is an illustrative diagram of a transducer 400 according to some embodiments. Transducer 400 includes a piezoelectric crystal/layer 420 with its top and bottom sides metal covered (e.g., sputtered), a backing layer 425 connected to either side of the piezoelectric layer 420, a matching layer 410, and a laterally surrounding protective cover or protective layer 405. The piezoelectric crystal 420 is constructed to mechanically vibrate in response to ultrasonic waves incident upon the transducer and, in response, generate a voltage across the piezoelectric crystal 420 (i.e., direct piezoelectric effect). This charge differential may be carried through a connected conductor. The variance in charge across the piezoelectric crystal 420 may be correlated with ultrasonic frequencies incident upon the piezoelectric crystal 420 over time (e.g., by the computer system 36). Similarly, an electrical charge may be introduced across the piezoelectric crystal 420 via connected conductors and an external electric power source and cause the piezoelectric crystal 420 to emit ultrasonic waves (i.e., inverse piezoelectric effect). Such an emission may be used to deliver ultrasound to external structures and media (e.g., vessel walls and blood), after which a responsive signal (e.g., echo signals) may be monitored to detect the presence, distances, dimensions, and characteristics of those structures and media.

In certain embodiments, the backing layer 425 (aka “damping block”) is included to absorb and damp extraneous emissions (“noise”) from the piezoelectric crystal 420 not directed toward targeted structure and media. The backing layer 425 may be substantially non-conductive so that it does not interfere with measuring signals across the piezoelectric crystal 420. In some embodiments, the backing layer 425 may be conductive and be utilized as an electrode. Without a backing layer 425, the noise from extraneous emissions can interfere with the detection of and accurate processing of return signals. This noise suppression is often needed for obtaining detailed measurements of the content and morphology of targeted structures from a transducer in linear and linear-phased transducer arrays as employed in many IVUS systems.

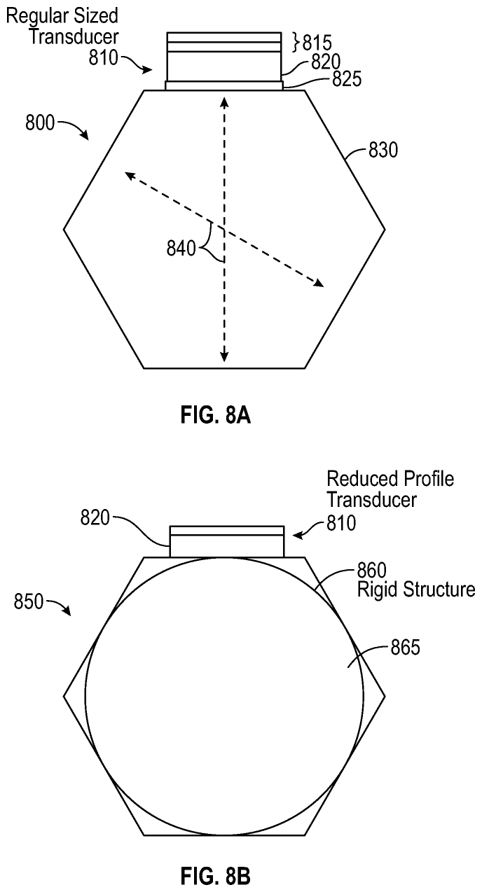

In some embodiments, a reduced thickness backing layer 425 has a thickness that produces −20 dB or lower of round-trip attenuation. In some embodiments, the reduced backing layer 425 is less than about half to about a tenth of the thickness of the piezoelectric crystal 420 to which it is attached. The effects of a reduced or omitted backing layer 425 may be addressed in various ways (e.g., by an expansive rigid body segment as shown and described in reference to FIGS. 8A-B or materials layered beneath the piezoelectric layer such as further described herein).

Matching layer 410 can be used to gradually transition or better “match” the acoustic impedances between the piezoelectric crystal 420 and the targeted structure, thereby improving the strength and detail of return signals from the imaged structure. In some embodiments, the matching layer 410 is substantially non-conductive in order to avoid interfering with measuring charge across the piezoelectric crystal 420. In some embodiments, the matching layer 410 is conductive (e.g., a conductive epoxy) and can also operate as an electrode for measuring charge across the piezoelectric crystal 420. In some embodiments, the conductive epoxy matching layer 410 has a thickness of less than about a quarter of a wavelength of a resonant frequency of the conductive epoxy material. In some embodiments, the matching layer 410 is a metallic conductive layer and has a thickness of less than about a quarter of a wavelength of a resonant frequency in the metallic conductive layer material. In some embodiments, the metallic conductive material is the conductive epoxy material. In some embodiments, the metallic conductive layer over the top side of the piezoelectric layer of each of the transducers is substantially only a malleable metal. In certain embodiments, the lateral protective layer 405 seals the transducer from external environmental factors and media and may also be configured to damp unwanted lateral-mode vibrations. In some embodiments, the protective layer 405 is substantially non-conductive, or may be omitted.

FIG. 4B is an illustrative diagram of a transducer according to some embodiments. Transducer 430 includes a piezoelectric crystal/layer 440 with its top and bottom sides metal covered (e.g., sputtered), a matching layer 435, and a protective layer 445. Transducer 430 omits a backing layer (e.g., the backing layer 425 of the transducer 400 of FIG. 4A). In some embodiments, the matching layer 435 has a thickness equal to or less than about a quarter of the resonant wavelength of the matching layer material. In some embodiments, the matching layer 435 is conductive and can include a conductive epoxy, for example. By omitting a backing layer and optionally reducing the thickness of the matching layer 435, the transducer 430 occupies less space and has a lower footprint than the transducer 400. The transducer 430 will thus likely generate greater noise than the transducer 400 that may obscure the pertinent signals pertaining to structural and morphological features.

FIG. 4C is an illustrative diagram of a transducer 450 according to some embodiments. The transducer 450 includes a piezoelectric crystal/layer 465 with its top and bottom sides metal covered (e.g., sputtered) and an additional electrode 470 directly positioned under the piezoelectric crystal 465. The transducer 450 omits a backing layer and a matching layer (e.g., backing layer 425 and matching layer 410 of FIG. 4A and/or matching layer 435 of FIG. 4B). By omitting a backing layer and a matching layer, transducer 450 occupies less space and has a lower footprint than the transducers 400 or 430. Along with likely generating greater noise by lacking a backing layer, the lack of a matching layer may reduce the transducer operating frequency bandwidth and may further increase noise as a result of the abrupt transition of acoustic impedance from the piezoelectric crystal 465 to external media.

In certain embodiments, the electrode 470 may be an electrode layer sputtered over a back (inner) side of the transducer 450 and/or first positioned on the surface of an imaging probe before the transducer 450 is placed on the electrode 470. The electrode 470 may alternatively be omitted or cover a reduced portion (e.g., less than about 10% of the surface) of the back (inner) side of the transducer 450. A connector (e.g., a flexible ribbon or wire connector 475) may be attached to the electrode 470 or top/bottom surface of the piezoelectric crystal 465 and connected to cables/connectors that extend to and/or are connected with a signal processor (e.g., data acquisition unit 34 of FIG. 1) during integration of the transducer 450 with a transducer array/imaging probe. In some embodiments, the connector 475 may form a ball, wedge, or other suitable form for adherence and appropriate mechanical strength. In some embodiments, a lateral protective layer (e.g., lateral protective layer 405) may be applied after connection of the connector 475 to the piezoelectric crystal 465 so that the connector 475 extends from the lateral protective layer 405.

In some embodiments, the electrode 470 is a conductive material configured and arranged to absorb/damp a portion of ultrasonic waves emitted in a direction away from the front/outer side of piezoelectric crystal 465. In some embodiments, the electrode 470 damps a substantial portion of such ultrasonic waves. In some embodiments, the electrode 470 has a thickness that produces −20 dB or lower of round-trip attenuation.

In some embodiments, the transducers of FIGS. 4A-4C are configured to operate independently as A-mode transducers (e.g., not as part of a linear or a linear-phased array of transducers operating as B-mode transducers). In some embodiments, the piezoelectric crystal/layers 420, 440, and 465 have a width to thickness ratio (“aspect ratio”) of at least about 0.6. In some embodiments, the width to thickness ratio is between about 2 and 4. In some embodiments, their aspect ratios are greater than about 4. In some embodiments, the aspect ratio is 1. In some embodiments, their aspect ratio is less than 20. In some embodiments, their aspect ratio is between 3 and 15. In some embodiments, their aspect ratio is between 2 and 7. In some embodiments, the width of the piezoelectric layer is at least the wavelength of the resonant frequency of an external environment outside of the catheter body. In some embodiments, the piezoelectric crystal/layers have a thickness of about one half of their resonant wavelength or slightly less (e.g., between about 0.4 and about 0.5 of the piezoelectric crystal/layer resonant wavelength).

In some embodiments, systems and methods are described herein that process transducer signals using the described transducers (e.g., from transducers with reduced or without conductive backing layers and/or matching layers such as transducers 450 and 430) to calculate distance measurements while taking advantage of the lower footprints of transducers 430 and 450. For example, transducers 450 and 430 may be integrated with the catheter systems and apparatus of FIGS. 1, 2A-C, 3A-D, 8A-B, 9A-B, 14A-C, 17A-B, 18A-C, and 19 to determine dimensional, structural, and morphological features of targeted structures including body lumens.

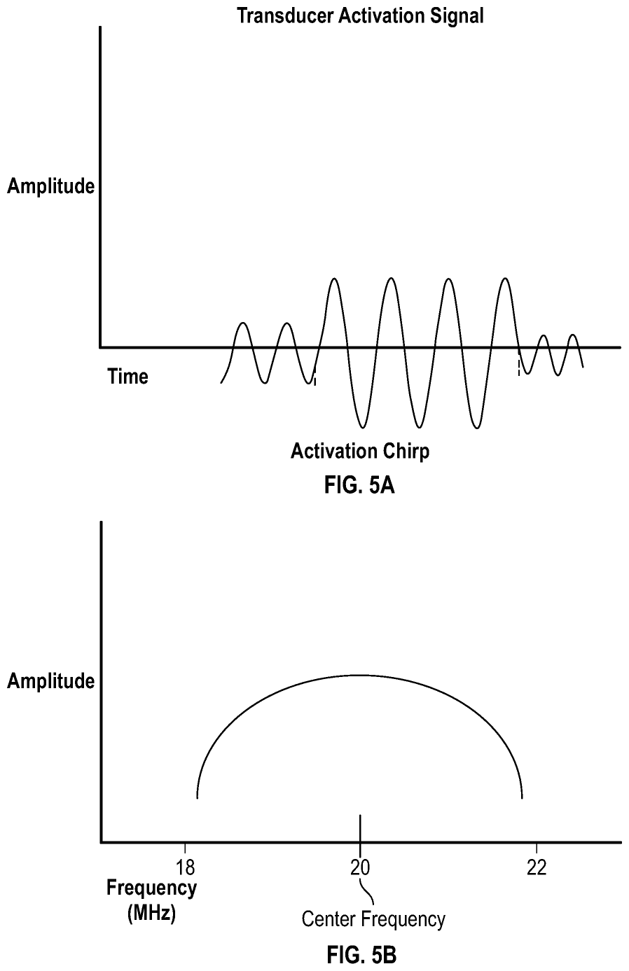

FIG. 5A is an illustrative chart of a pulse (“chirp”) for activating a transducer according to some embodiments. In some embodiments, the pulse or chirp represents a modulated pulse (of electronic signals) having a frequency centered about the same resonant frequency of the transducer to be activated. In some embodiments, the chirp represents an envelope pulse of increasing and decreasing signal frequency and amplitude centered at a particular frequency (e.g., the resonant frequency of a transducer).

FIG. 5B is an illustrative chart of the frequency spectrum of a pulse for activating a transducer according to some embodiments. In some embodiments, the frequency of the signals gradually increases to and peaks at the transducer's resonant frequency and gradually decreases before terminating, determining the transducer's operating frequency bandwidth.

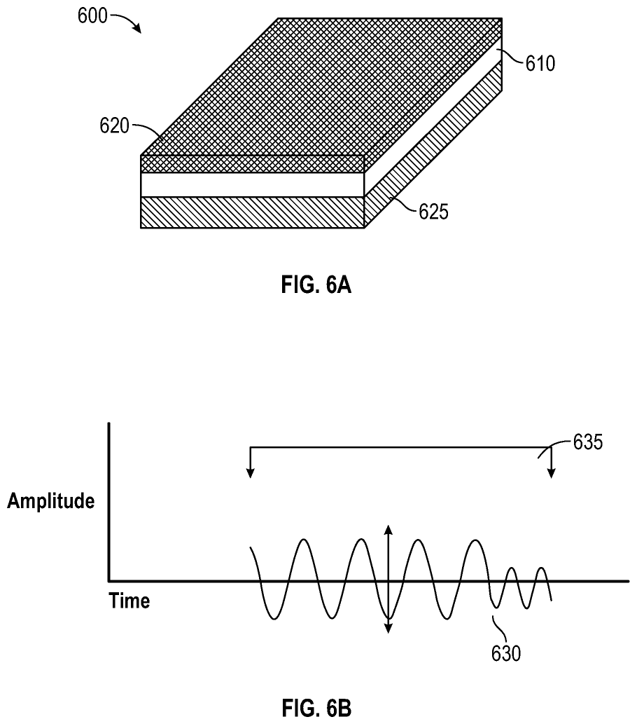

FIG. 6A is an illustrative diagram of a transducer 600 according to some embodiments. The transducer 600 includes a piezoelectric crystal/layer 610 with its top and bottom sides metal covered (e.g., sputtered), a backing layer 625, and a matching layer 620. The piezoelectric crystal 610 is constructed to mechanically vibrate in response to ultrasonic waves incident upon the transducer and, in response, generate a voltage across the piezoelectric crystal 610 (i.e., direct piezoelectric effect). This charge differential may be carried through a connected conductor. The variance in charge across the piezoelectric crystal 610 may be correlated with ultrasonic frequencies incident upon the piezoelectric crystal 610 over time (e.g., by the computer system 36). Similarly, an electrical charge may be introduced across the piezoelectric crystal 610 via connected conductors and an external electric power source and cause the piezoelectric crystal 610 to emit ultrasonic waves (i.e., inverse piezoelectric effect). Such an emission may be used to deliver ultrasound to external structures and media, after which a responsive signal (e.g., echo signals) may be monitored to detect the presence and characteristics of those structures and media.

Backing layer 625 (aka “damping block”) is included to absorb and damp extraneous emissions (“ringing noise”) from the piezoelectric crystal 610 not directed toward targeted structure and media. The backing layer 625 may be substantially non-conductive so that it does not interfere with measuring signals across the piezoelectric crystal 610. In some embodiments, the backing layer 625 may be conductive and be utilized as an electrode. Without a backing layer 625, the noise from extraneous emissions can interfere with the detection of and accurate processing of return signals.

Noise suppression is often needed for obtaining detailed measurements of the content and morphology of targeted structure from a transducer as employed in many IVUS systems. In some embodiments, the backing layer 625 has a thickness that produces −20 dB or lower of round-trip attenuation. In some embodiments, the backing layer 625 is less than about half to about a tenth of the thickness of the piezoelectric crystal 610 to which it is attached. The effects of a reduced or omitted backing layer may be addressed in various ways (e.g., by a reinforced hardened body segment as shown and described in reference to FIGS. 8A-8B or materials layered beneath the piezoelectric layer such as further described herein). In some embodiments, the backing layer 625 is comprised substantially of a material having a Shore hardness of at least about 70 D.

Matching layer 620 can be used to gradually transition or better “match” the acoustic impedances between the piezoelectric crystal 610 and the targeted structure, thereby improving the strength and detail of return signals from the imaged structure. In some embodiments, the matching layer 620 is substantially non-conductive in order to avoid interfering with measuring charge across the piezoelectric crystal 610. In some embodiments, the matching layer 620 is conductive (e.g., a conductive epoxy) and can also operate as an electrode for measuring charge across the piezoelectric crystal 610. A protective layer (e.g., coating 315 of FIG. 3C) may be used to seal the transducer 600 from external environmental factors and media and may also be configured to damp and/or provide “matching” characteristics similar to the matching layer 620. In some embodiments, the protective layer is substantially non-conductive, or may be omitted.

FIG. 6B is an illustrative chart of a response over time to a pulse of determined frequency in the transducer 600 of FIG. 6A. In a traditional transducer with full sized backing and matching layers, an initial pulse 630 generated after being activated will have a limited amplitude and relatively shorter “ring down” period 635. If the backing layer is reduced or omitted (e.g., as shown in FIG. 7A), the initial pulse 630 will have a relatively larger amplitude and longer period of ring down. A transducer 600 with a backing layer to absorb errant reverberations will generally produce an initial signal envelope over a relatively short period 635 of time, typically damped out by the time return signals representing externally imaged features are received. The matching layer 620 will further reduce noise caused by mismatched acoustic impedances between the piezoelectric crystal 610 and externally imaged features. Additionally, the matching layer 620 will provide an increased operating frequency bandwidth at which the piezoelectric crystal 610 can be activated either by an applied electrical charge or returning ultrasonic waves.

FIG. 7A is an illustrative diagram of a transducer 700 for integration into an imaging probe transducer array according to some embodiments. The transducer 700 includes a piezoelectric crystal/layer 710 with its top and bottom sides metal covered (e.g., sputtered), but does not include a backing layer or matching layer. Prior to integration into an imaging probe, the transducer 700 may include an attached electrode 720 and a connector 725 for obtaining ultrasound signals that produce voltage differences across the piezoelectric crystal 710.

FIG. 7B is an illustrative chart of a response over time to a pulse of determined frequency in a transducer (e.g., transducer 700 of FIG. 7A) with a reduced or omitted backing layer according to some embodiments. The lack of a backing layer may cause generation of an initial signal envelope 730 with higher amplitude and over a relatively longer (undamped) period 735 of time compared to period 635 for a transducer 600 having a backing layer 625. Lack of a matching layer may decrease the operating frequency bandwidth at which the piezoelectric crystal 710 can be activated either by an applied electrical charge or returning ultrasonic waves. Lack of a matching layer may also increase noise. This increased noise may overlap and interfere with signals received that represent imaged features. In various embodiments described herein, methods and systems of integration and operation adapt to increased noise generated from such transducers for imaging external features while reducing the footprint of an ultrasound probe with multiple such transducers. In some embodiments, signals or characteristics of these signals associated with such reverberations are learned such as by using machine learning techniques. The learned signals (i.e., noise) may then later be compared with signals including those representing imaged features and subtracted or distinguished from the overall signal to identify the imaged features. The subtracted/distinguished signal of the imaged features may then be used to calculate distance between an imaging probe and the features (e.g., a vessel wall) such as by using time-of-flight (TOF) information and known information about the location of the transducer within the imaging probe.

FIG. 8A is an illustrative cross-sectional diagram of a transducer 810 positioned on a probe body segment 830 according to some embodiments. A transducer assembly 800 includes the transducer 810, which includes a piezoelectric layer 820 and may include a reduced (or omitted) backing layer 825 and/or reduced (or omitted) matching and/or protective layers 815 such as described in reference to FIGS. 4A-4C, 6A, and 7A, for example. The transducer 810 is placed/positioned on the probe body segment 830, which may be manufactured/assembled to be of a hardened/rigid material and extent 840 so as to attenuate ultrasound signals directed toward the central longitudinal axis of a catheter body (e.g., catheter body 40 of FIG. 1) and to direct ultrasound signals away from the central longitudinal axis of the catheter body. In some embodiments, the diameter/circumscribed diameter of the probe body segment 830 is between about 500 μm and 2 mm. In some embodiments the diameter/circumscribed diameter of the probe body segment 830 is about 1 mm. In some embodiments, the probe body segment 830 is assembled to encompass the central longitudinal axis of a catheter body. In some embodiments, the probe body segment 830 is a solid body that substantially incorporates a material having a Shore hardness of at least about 65 D. Other adjacent segments of the probe/catheter may be selected to provide sufficient flexibility for the particular application and can be substantially less stiff than the probe body segment 830 (e.g., a coronary catheter may require a high degree of flexibility). In some embodiments, the probe body segment 830 is configured to be radiopaque and permit an imaging device (e.g., radiography device) to locate its position within a structure.

In some embodiments, the probe body segment 830 represents a polygon. In some embodiments, the transducer 810 is positioned on one outer edge/side of the polygon. In some embodiments, each of a plurality of transducers 810 individually occupies an outer edge/side of the polygon. In some embodiments, the polygon has about 5 to 16 edges/sides and may have a corresponding transducer 810 on each edge/side.

FIG. 8B is an illustrative cross-sectional diagram of a transducer 810 positioned on a probe body 860 according to some embodiments. A transducer assembly 850 includes the transducer 810 that is positioned on the probe body 860 within which an inner segment 865 is integrated. The inner segment 865 may be a ring or cylinder, for example, and comprise metal and/or other hard materials so as to resist acoustic wave transmission from a piezoelectric layer 820 toward the probe body 860 while not substantially impacting the overall flexibility of the probe body 860. In some embodiments, the inner segment 865 is configured to be radiopaque. The probe body 860 may be a segment or portion of a catheter body (e.g., similar to segment 830) and reinforced by the inner segment 865 to increase overall resistance to internal transmission of acoustic waves, to attenuate acoustic waves directed toward the central longitudinal axis of a catheter/probe, and to direct acoustic waves away from the central longitudinal axis of a catheter/probe. Probe body segments 830, 860, and/or inner segment 865 may be incorporated with an array of transducers (e.g., the arrays shown in FIGS. 1, 2A-C, 3A-D, 8A-B, 9A-B, 14A-C, 17A-B, 18A-C, and 19), each of which may operate independently as A-mode transducers.

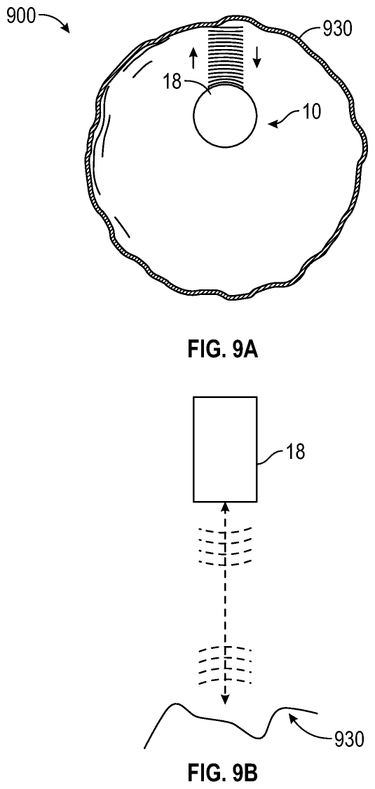

FIG. 9A is an illustrative diagram of an imaging probe 10 inserted into a lumen or blood vessel 900 according to some embodiments. FIG. 9B is an illustrative diagram of an ultrasound transducer 18 of FIG. 9A imaging a lumen wall 930. The imaging probe 10 (e.g., as shown in FIG. 1) includes the transducer 18 which can be one of an array of A-mode/independent transducers such as described herein. One transducer 18 is illustrated generating and receiving ultrasound signals that are incident on the wall 930 of the blood vessel 900 and echo back to the transducer 18.

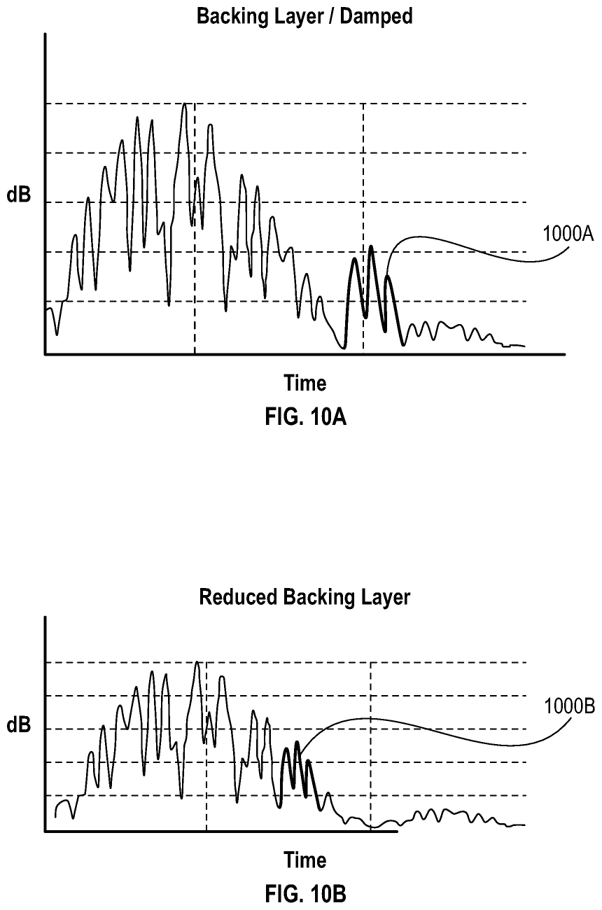

FIG. 10A is an illustrative chart of a frequency response in an ultrasound transducer having a backing layer. When a transducer 18 includes a backing layer and/or matching layer (e.g., as shown in FIGS. 4A and 6A) a signal response 1000A representing imaged features may likely be distinct and well separated from noise representing the initial pulse signal reverberating in the transducer 18.

FIG. 10B is an illustrative chart of a frequency response in the transducer 18 without all or a portion of a backing and/or matching layer according to some embodiments (e.g., as shown in FIGS. 4B and 7A). On account of omitting all or portions of a backing and/or matching layer, a predominant portion of the signal may be attributed to the initial activation of the transducer 18; in which case, a signal response 1000B representing imaged features may likely not be distinct and well separated from the initial pulse signal reverberating in the transducer 18. In some embodiments described further herein, signals attributed to a lumen wall (e.g., peaks at 1000A and 1000B) are identified and extracted from their respective signals. This may be performed, for example, by comparing the signal generated without a lumen wall adjacent the probe to a signal with a lumen wall adjacent the probe.

In some embodiments, signals or characteristics of these signals associated with such reverberations are learned such as by using machine learning techniques. The learned signals (i.e., noise) may then later be compared with signals including those representing imaged features and subtracted or distinguished from the overall signal to identify the imaged features. The subtracted/distinguished signal of the imaged features may then be used to calculate distance between an imaging probe and the features (e.g., a lumen wall) such as by using time-of-flight (TOF) information and known information about the location of the transducer within the imaging probe.

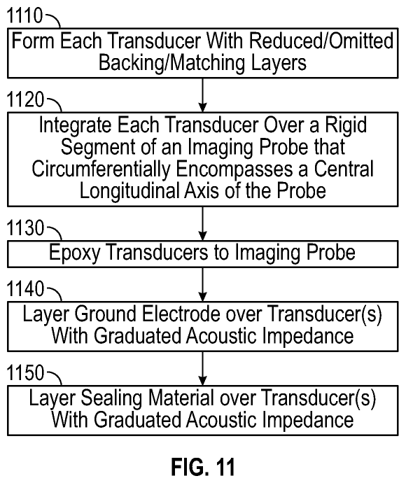

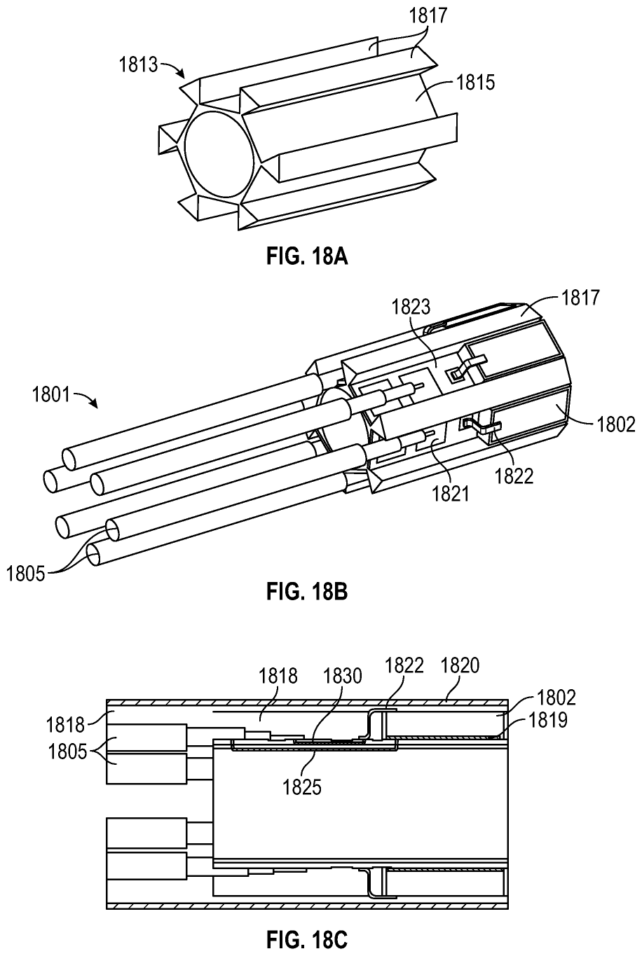

FIG. 11 is an illustrative flowchart of a process for assembling an imaging probe according to some embodiments. At block 1110, a signal electrode is attached to the back side of a piezoelectric layer to form each of a plurality of transducers. In some embodiments, a backing layer, protective layer, and/or matching layer is omitted from the formed transducers such as described in reference to FIGS. 4B-C and 7A. At block 1120, each of the transducers is then placed into a form-fitting cavity of an imaging probe such as the cavities/slots 1815 of FIG. 18A.

At block 1130, epoxy or other adherent/sealant is applied around at least the edges of each of the transducers to adhere/seal them within each of their respective cavities. Epoxy can further be applied around the transducers to provide a smooth edge across surfaces of the imaging probe.

After application of the adherent/sealant at block 1130, a ground electrode may be applied to the top/front side of each of the transducers at block 1140. In some embodiments, a layer of electrode material is formed over the transducers. The layer may be formed by sputtering the material over the transducers and can be a common/connected layer among the transducers. In some embodiments, the ground electrode comprises metal including, for example, gold, copper, chrome, titanium, brass, silver, and/or platinum. In some embodiments, the ground electrode is comprised substantially of metal. The electrode material may be formed from other materials such as conductive epoxies, for example.

After application of the ground electrode at block 1140, a sealing layer is applied at block 1150 to further seal the transducers from external environmental factors. The sealing layer can be, for example, a biocompatible material that permits easy movement of the imaging probe within a lumen such as a blood vessel. The sealing layer may be applied directly on the ground electrode. As further described herein, the ground electrode and sealing layer may be formed to gradually transition the acoustic impedance between the transducers and materials/media that are to be imaged. In some embodiments, the positions of the ground and signal electrodes are reversed between the top/front and bottom/back sides of the transducers.

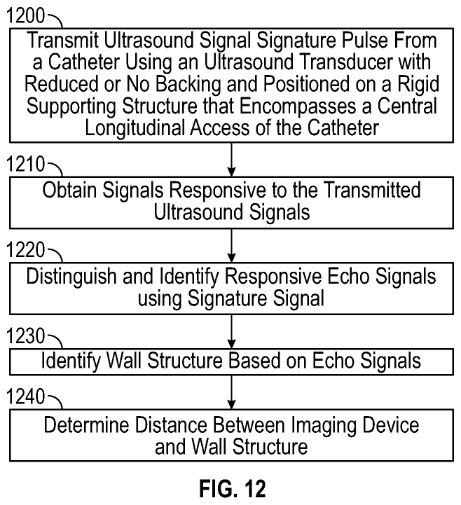

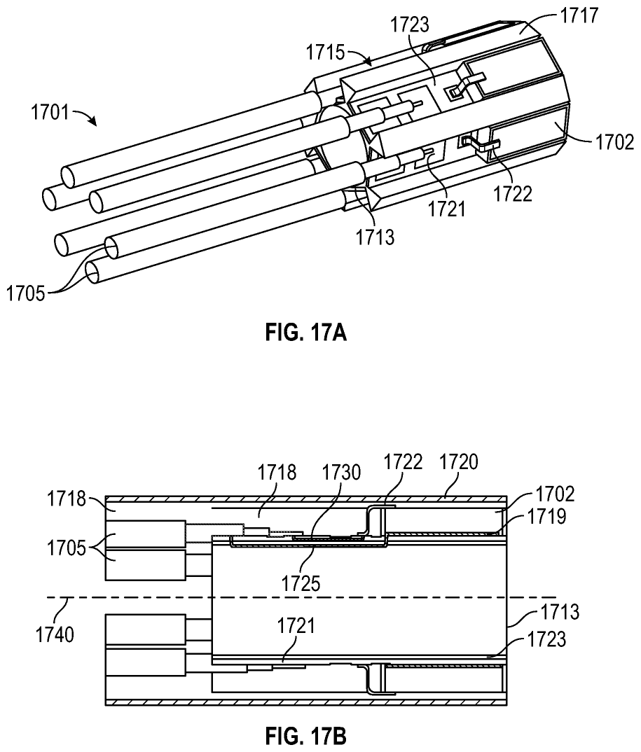

FIG. 12 is an illustrative flowchart of a process for processing signals from a transducer with a reduced or omitted backing and/or matching layer according to some embodiments. At block 1200, an ultrasound signal is generated/transmitted from the transducer, which is integrated into an imaging device (e.g., imaging probe 10 of FIG. 1) according to some embodiments. At block 1210, signals responsive to the ultrasound signal transmitted at block 1200 are received back at the transducer and/or other transducers (e.g., in a transducer array such as shown in FIGS. 17A-17B).

At block 1220, the responsive signals received at block 1210 are analyzed to distinguish signals of targeted features from noise (e.g., signals associated with reverberations in the transducer from the signal generated at block 1200). In some embodiments, the targeted features include the boundary of a blood vessel wall, for example. The signals associated with noise may be determined, for example, based on learning to identify (e.g., using known machine learning techniques) and distinguish noise from previously obtained signals using the same or a similar transducer.

At block 1230, based on the analysis at block 1220, a wall structure is identified from the responsive signals. A wall structure may be identified such as by eliminating identified noise from the signal and/or using machine learning techniques based on signals of similar wall structures previously imaged/learned. At block 1240, based on identifying the wall structure at block 1230, a distance is determined between the imaging device and the wall structure. This distance may be calculated, for example, by using TOF information pertaining to the wall structure signal. This can be performed, for example, by timing the interval between when a pulse is delivered and when the signal associated with the wall is received by the transducer and knowing information about the speed of ultrasonic waves in the medium (e.g., blood) between the transducer and wall structure. Based on determining this distance and/or other similarly determined distance calculations between other transducers of the imaging device and wall structure, a cross-sectional mapping or shape of the wall structure can be further determined such as described in the '701 patent.

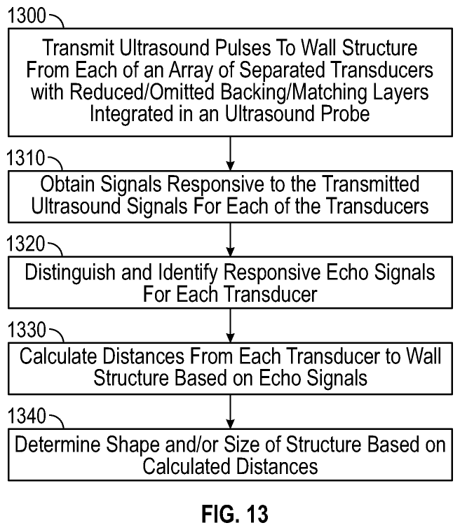

FIG. 13 is an illustrative flowchart of a process for measuring a structure using an ultrasound transducer array according to some embodiments. At block 1300, an ultrasound signal (i.e., a pulse) is generated/transmitted from each transducer of an array of transducers and directed to the structure. In some embodiments, the transducers are configured with reduced/omitted backing and/or matching layers such as further described herein and/or are separated (e.g., circumferentially) by a center-to-center pitch of at least about twice the wavelength of the ultrasound signals in the medium (e.g., blood) in which they are transmitted. At block 1310, signals responsive to the ultrasound signal transmitted at block 1300 are received back at the transducer and/or other transducers (e.g., in a transducer array such as shown in FIGS. 1, 2A-C, 3A-D, 8A-B, 9A-B, 14A-C, 17A-B, 18A-C, and 19).

At block 1320, the responsive signals received at block 1310 are analyzed to distinguish signals of targeted features from noise (e.g., signals associated with reverberations in the transducer from the signal generated at block 1300). In some embodiments, the identified targeted features include the boundary of a blood vessel wall, for example. The signals associated with noise may be determined, for example, based on learning to identify (e.g., using known machine learning techniques) and distinguish noise from previously obtained signals using the same or a similar transducer.

At block 1330, based on the analysis at block 1320, a distance is determined between the imaging device and the identified wall structure. This distance may be calculated, for example, by using TOF information pertaining to the wall structure signal. This can be performed, for example, by timing the interval between when a pulse is delivered and when the signal associated with the wall is received by the transducer and knowing information about the speed of ultrasonic waves in the medium (e.g., blood) between the transducer and wall structure.

At block 1340, based on determining the distances and/or other similarly determined distance calculations between each of the transducers and wall structure, a cross-sectional mapping or shape of the wall structure can be further determined such as described in the '701 patent. Mappings/shapes may be used to calculate diameters, areas, volumes and/or other features of the structure (e.g., of a blood vessel lumen).

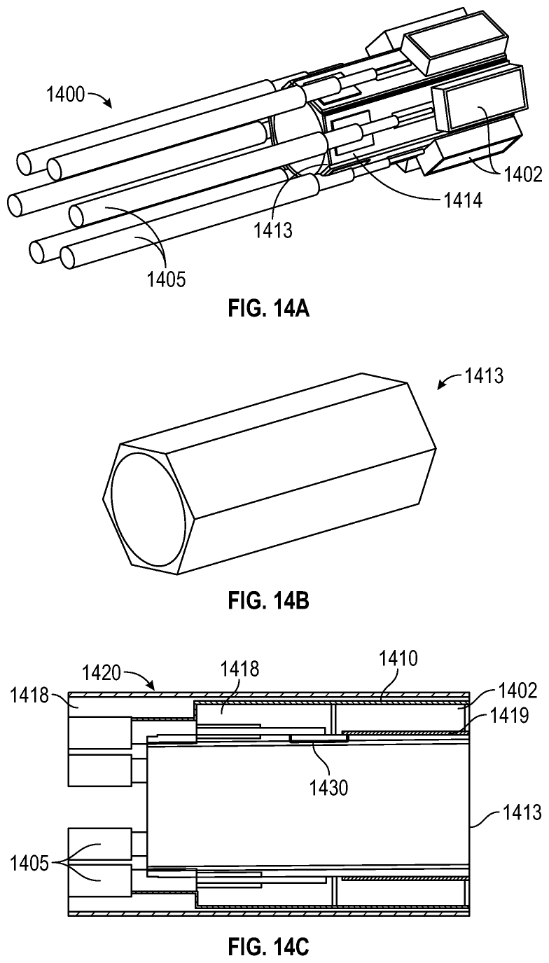

FIG. 14A is an illustrative diagram of a transducer array integrated with a probe body according to some embodiments. FIG. 14B is an illustrative diagram of a probe body according to some embodiments. FIG. 14C is an illustrative cross-sectional diagram of a transducer array integrated with a probe body according to some embodiments. Transducer array 1400 includes transducers 1402 integrated with a probe body 1413. Probe body 1413 is hexagonal in which its hexagonal surfaces are substantially flat and of a width conformed to the size of transducers 1402. In some embodiments, depending on the number of transducers in an array, the number of sides of body 1413 may be correspondingly adjusted. Transducers 1402 are positioned on the surfaces of probe body 1413 and connected with transducer conductors/cables 1405 that can extend along the length of an imaging probe to a signal processor/analyzer. In some embodiments, transducers 1402 are connected to or mounted on flexible circuits 1414, that can be single or multi-layer structures of flexible polymers (such as but not limited to polyimides) and metals (such as but not limited to gold sputtered copper).

An electrode 1419 (e.g., a signal/ground electrode) may be a component of a transducer 1402 (e.g., electrode 720 of FIG. 7A) and/or separately positioned on flexible circuit 1414 mounted on probe body 1413. In some embodiments, electrodes 1419 connect to ground/signal of respective connectors/cables 1405 via connectors/conductors 1430 (interlayer metal traces connected through metal plated vias). An epoxy/sealant 1418 may be placed around or proximate to transducers 1402 in order to seal/adhere them or align their surfaces with respect to other portions (e.g., an outermost surface) of the transducer array 1400. A ground electrode 1410 is applied over the transducers and may be applied as a common layer (e.g., sputtered) over all of the transducers of the array. An insulating layer 1420 is positioned over the transducers to insulate them from external factors and/or to provide biocompatibility with structures in which the array 1400 is inserted.

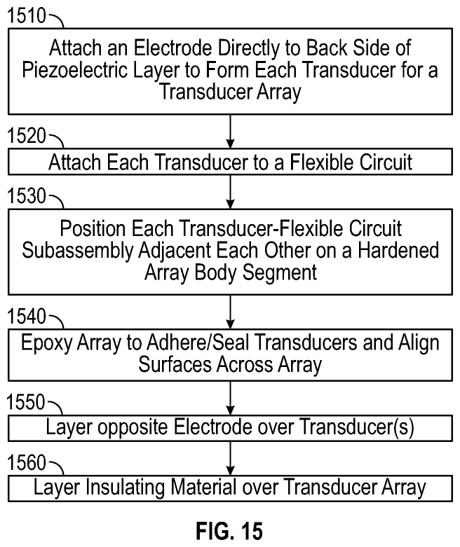

FIG. 15 is an illustrative flowchart of a process for assembling a transducer array probe according to some embodiments. At block 1510, a signal electrode is attached to the back side (or back-facing side) of each transducer of a transducer array. The signal electrodes may be layered upon or otherwise attached to the back sides of each transducer. At block 1520, each transducer is attached to a flexible circuit (e.g., flexible circuit 1414 of FIG. 14A using conductive epoxy). At block 1530, each transducer-flexible circuit subassembly is positioned adjacent each other on an array body (e.g., probe body 1413 of FIG. 14B). These subassemblies may be attached with non-conductive epoxy or otherwise affixed to the flat surfaces of probe body 1413.

At block 1540, epoxy or other suitable adherent/sealant is applied to the areas between the transducers and other surfaces to adhere/seal the transducers to the array and align surfaces (e.g., outermost surfaces) across and around the array (e.g., to provide an even/smooth surface across a probe). At block 1550, an opposite ground or signal electrode is applied (e.g., laminated or sputtered) to the top (or front side) of each of the transducers. In some embodiments, a common/connected layer of electrode material is formed over multiples of the transducers. The layer may be formed by sputtering the electrode material (e.g., an adhesion layer of chrome followed by a layer of gold, copper, and/or platinum, whose thicknesses depend on the resonant frequency of the transducers). This common ground/signal electrode can be formed in other ways such as by application of a conductive epoxy (e.g., silver particles mixed in hardener resin). At block 1560, an insulating material is applied over the transducer array to seal it from environmental/external factors and/or to provide a biocompatible surface to the array. This insulating material can be made of polymers, such as but not limited to vapor deposited parylene, whose thickness may be adjusted equal to or less than about one quarter of the resonant wavelength in the polymer material to gradually transition the acoustic impedance to the medium (e.g., blood) and the structure being imaged (e.g. blood vessel wall).

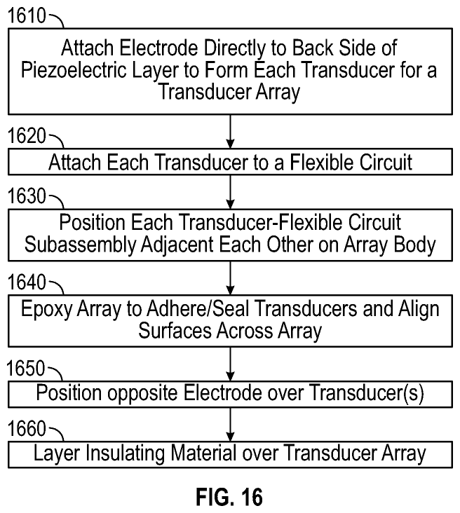

FIG. 16 is an illustrative flowchart of a process for assembling a transducer array probe according to some embodiments. At block 1610, an electrode (e.g., a signal or ground electrode) is attached to the back side (or back-facing side) of each transducer of a transducer array. The signal electrodes may be layered upon or otherwise directly attached to the back sides of each transducer. At block 1620, each transducer is attached to a flexible circuit (e.g., flexible circuit 1723 of FIGS. 17A-B) with conductive epoxy. At block 1630, each transducer-flexible circuit subassembly is positioned adjacent each other on an array body (e.g., probe body 1713 of FIGS. 17A-B). These subassemblies may be attached with non-conductive epoxy or otherwise affixed to the flat surfaces of a probe body.

At block 1640, epoxy or other suitable adherent/sealant is applied to the areas between the transducers and other surfaces to adhere/seal the transducers to the array and align surfaces (e.g., outermost surfaces) across and around the array (e.g., to provide an even/smooth surface across a probe). At block 1650, a signal/ground electrode is applied to the top (or front side) of each of the transducers. In some embodiments, a common/connected layer of electrode material is formed over multiples of the transducers. The layer may be formed by sputtering the electrode material (e.g., an adhesion layer of chrome followed by a malleable layer of metal (e.g., gold, copper, and/or platinum), whose thicknesses may depend on the resonant frequency of the transducers so as to gradually transition acoustic impedance to media/structure outside of the transducers). This common signal/ground electrode can be formed in other ways such as by application of a conductive epoxy (e.g., silver particles mixed in hardener resin). The signal/ground electrode of each of the transducers may additionally take the form of a ribbon, wire, wedge, and/or ball (e.g., ribbon connector 1722 of FIGS. 17A-B). At block 1660, an insulating material is applied over the transducer array to seal it from environmental/external factors and/or to provide a biocompatible surface to the array. This insulating material can be made of polymers, such as but not limited to vapor deposited parylene, whose thickness may be adjusted equal to or less than about one quarter of the resonant wavelength in the polymer material to gradually transition the acoustic impedance to the medium (e.g., blood) and the structure being imaged (e.g. blood vessel wall).

FIG. 17A is an illustrative diagram of an ultrasound probe 1701 integrated with a probe body according to some embodiments. FIG. 17B is an illustrative cross-sectional diagram of the ultrasound probe 1701 of FIG. 17A according to some embodiments. A probe body 1713 includes slots (or cavities) 1715 that are form-fitted to receive/fit transducers 1702 within them. The slots 1715 are formed by columns (or protruding ridges) 1717.

In some embodiments, the probe body 1713 is manufactured/assembled to be of a hardened/rigid material and extent so as to attenuate ultrasound signals directed toward the central longitudinal axis 1740 of ultrasound probe 1701 and to direct ultrasound signals away from the central longitudinal axis 1740.

In some embodiments, each of the columns 1717 are angled on their side walls to converge inward toward each other from an outer surface of the columns. In some embodiments, the side walls or other features of transducers 1702 are correspondingly shaped and positioned to hold and/or position the transducers in slots 1715. During assembly of the transducer array, the transducers may be inserted/slid along a longitudinal axis of body 1713 through a slot 1715 into a desired longitudinal position where it can be further affixed to body 1713.

Transducer connectors/cables 1705 connect and extend between transducers 1702 and a signal processor (e.g., in data acquisition unit 34 of FIG. 1). In some embodiments, flexible conductive ribbon connectors 1722 are positioned to electrically connect the outer surfaces of transducers 1702 to ground/signal of connectors/cables 1705 via connectors/conductors 1730 (interlayer metal traces connected through metal plated vias). Electrodes 1721 on flexible circuits 1723 can be electrically connected to the back/inner sides of transducers 1702 such as through signal/ground electrode surfaces 1719 and connectors/conductors 1725 (interlayer metal traces connected through metal plated vias) attached to the back/inner sides of transducers 1702.

An epoxy/sealant 1718 may be placed around or proximate to transducers 1702 as a lateral protective layer in order to seal/adhere them or align their surfaces with respect to other portions of the ultrasound probe 1701. These lateral layers may be configured to direct ultrasound signals from transducers 1702 away from longitudinal axis 1740 and to damp excessive lateral-mode ringing of the piezoelectric elements of the transducers. In some embodiments, the lateral protective layering retains a flexibility sufficient to permit transducers 1702 to generate a sufficient external (i.e., in radially outward direction) acoustic signal for purposes of measurement. In some embodiments, the lateral protective layering has a hardness of between about 20 D and 90 D Shore hardness. In some embodiments, the lateral protective layer has a width of about a tenth to about a third of the thickness of the piezoelectric layer. In some embodiments, a tensile modulus of the lateral protective layer is between about 20 to 2500 N/mm\hat{ }2. An insulating material 1720 is applied over the transducer array to seal it from environmental/external factors and/or to provide a biocompatible surface to the probe 1701. In some embodiments, the lateral protective layer is omitted.

FIG. 18A is an illustrative diagram of a probe body according to some embodiments. FIG. 18B is an illustrative diagram of a transducer array integrated with the probe body of FIG. 18A according to some embodiments. FIG. 18C is an illustrative cross-sectional diagram of a transducer array integrated with a probe body according to some embodiments. A probe body 1813 includes slots (or cavities) 1815 that are form-fitted to receive/fit transducers 1802 within them. The slots 1815 are formed by columns (or protruding ridges) 1817.

In some embodiments, each of the columns 1817 are angled on their side walls to converge inward toward each other from an outer surface of the columns. In some embodiments, the side walls or other features of transducers 1802 are correspondingly shaped and positioned to hold and/or position the transducers in slots 1815. During assembly of the transducer array, the transducers may be inserted/slid along a longitudinal axis of body 1813 through a slot 1815 into a desired longitudinal position where it can be further affixed to body 1813.

Transducer connectors/cables/waveguides 1805 connect and extend between transducers 1802 and a signal processor (e.g., signal processor 34 of FIG. 1). In some embodiments, flexible conductive ribbon connectors 1822 are positioned to electrically connect the outer surfaces of transducers 1802 to ground/signal of the connectors/cables/waveguides 1805 via connectors/conductors 1830 (interlayer metal traces connected through metal plated vias). Ribbon connectors 1822 may also be formed as ball, wedge, or wire connectors, for example. Signal or ground electrodes 1821 on flexible circuits 1823 can be electrically connected to the back/inner sides of transducers 1802 such as through signal/ground electrode surfaces 1819 and connectors/conductors 1825 (interlayer metal traces connected through metal plated vias) attached to the back/inner sides of transducers 1802. In some embodiments, prior to attaching ribbon connectors 1822, a layer of a malleable metal (e.g., chrome, gold, copper, and/or platinum) is applied (e.g., laminated or sputtered) to the top/outer sides of transducers 1802. In some embodiments, the malleable metal layer has a thickness of at least about 0.5 microns.

An epoxy/sealant 1818 may be placed around or proximate to transducers 1802 in order to seal/adhere them or align their surfaces with respect to other portions of the transducer array 1801. An insulating material 1820 is applied over the transducer array to seal it from environmental/external factors and/or to provide a biocompatible surface to the array 1801.

FIG. 19 is an illustrative diagram of an angioplasty catheter integrated with an array of transducers according to some embodiments. A catheter 1900 includes an array of transducers 1918 that, when integrated into catheter 1900, include reduced/omitted backing and/or matching layers (e.g., like transducers of FIGS. 6A and/or 7A). After positioning on or into catheter 1900, these transducers may be integrated with the catheter with electrodes, adhesives, and/or sealing elements such as further described herein. Transducers 1918 are connected to a data acquisition and processing system (e.g., data acquisition unit 34 and processing unit 36 of FIG. 1) through a connector assembly 1935.

Catheter 1900 includes an expandable balloon 1925 (e.g., an angioplasty balloon) which can be expanded or deflated by controlling the introduction or expulsion of a medium (e.g., air or saline) through a lumen 1930. In some embodiments, readings from transducers 1918 are utilized to position balloon 1925 in an optimal location for deploying the balloon 1925 (e.g., within a diseased body vessel) and to center or hold catheter 1900 in a particular location.

Transducers 1918 may be operated such as described with respect to FIG. 13 to determine the distance and shape of surrounding blood vessels. In some embodiments, transducers 1918 are located within balloon 1925. These transducers may be used, for example, to monitor the level of expansion of balloon 1925. Balloon 1925 may be made of a material or include a coating that enhances their ultrasound reflectivity. The enhanced ultrasound reflectivity may be used, for example, to discriminate between added noise caused by a lack of backing and/or matching layers of transducers 1918 and signals representing balloon 1925.

The processes described herein (e.g., the processes of FIGS. 11, 12, 13, 15, and 16) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (for example, in the processes of FIGS. 11, 12, 13, 15, and 16) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

The processes described herein are not limited to the specific examples described. For example, the process of FIGS. 11, 12, 13, 15, and 16 are not limited to the specific processing orders illustrated. Rather, any of the processing blocks of FIGS. 11, 12, 13, 15, and 16 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any suitable sub-combination. Other embodiments not specifically described herein are also within the scope of the following claims.

1. An ultrasound transducer assembly comprising:
a piezoelectric layer configured to resonate and generate ultrasound signals around a predetermined ultrasound frequency, wherein the piezoelectric layer has a width to thickness ratio of at least about 0.6;
a conductive backing layer connected to the piezoelectric layer; and
a rigid body over which the conductive backing layer is positioned, the rigid body assembled for encompassing a central longitudinal axis of a catheter body,
wherein a rigidity of the rigid body is configured to attenuate ultrasound signals directed toward the central longitudinal axis of the catheter body and to direct ultrasound signals away from the central longitudinal axis of the catheter body.

2. (canceled)

3. (canceled)

4. The ultrasound transducer assembly of claim 1, wherein the width of the piezoelectric layer is at least about a wavelength of a resonant frequency of an external environment outside of the catheter body.

5. The ultrasound transducer assembly of claim 1, wherein the thickness of the piezoelectric layer is equal to or less than about one half a wavelength of a resonant frequency of the piezoelectric layer material.

6. The ultrasound transducer assembly of claim 1, wherein the width to thickness ratio of the piezoelectric layer is less than about 20.

7. (canceled)

8. The ultrasound transducer assembly of claim 1, wherein the rigid body is comprised substantially of a material having a Shore hardness of at least about 65 D.

9. The ultrasound transducer assembly of claim 1, wherein the conductive backing layer is configured to provide about −20 dB or less of round-trip attenuation.

10. (canceled)

11. The ultrasound transducer assembly of claim 1, wherein the conductive backing layer has a thickness of about a tenth to about a half of the thickness of the piezoelectric layer.

12. (canceled)

13. (canceled)

14. (canceled)

15. The ultrasound transducer assembly of claim 1, further comprising a metallic conductive layer over a top side of the piezoelectric layer, the metallic conductive layer configured to operate as a signal or ground layer.

16. The ultrasound transducer assembly of claim 15, wherein the metallic conductive layer is comprised substantially of a conductive epoxy onto which a malleable metal is applied, and wherein the malleable metal has a thickness of at least about 0.5 microns.

17. The ultrasound transducer assembly of claim 1, wherein a ribbon, ball, wedge, or wire electrode connector is attached to the conductive backing layer in order to carry a current between the piezoelectric layer and an external electrode.

18. (canceled)

19. (canceled)

20. (canceled)

21. The ultrasound transducer assembly of claim 15, wherein the metallic conductive layer is configured to transition acoustic impedances between the piezoelectric layer and an external environment outside of the catheter body.

22. The ultrasound transducer assembly of claim 1, further comprising an outermost protective layer over the piezoelectric layer and configured to transition an acoustic impedance between the piezoelectric layer and an external environment outside of the catheter body.

23. The ultrasound transducer assembly of claim 1, further comprising a lateral protective layer about lateral side surfaces of the piezoelectric layer, the lateral protective layer configured to suppress lateral-mode vibrations and to direct the ultrasound signals from the piezoelectric layer away from the central longitudinal axis of the catheter body.

24. (canceled)

25. (canceled)

26. (canceled)

27. (canceled)

28. The ultrasound transducer assembly of claim 23, wherein the lateral protective layer has a width of about a tenth to about a third of the thickness of the piezoelectric layer.

29. (canceled)

30. (canceled)

31. (canceled)

32. (canceled)

33. (canceled)

34. (canceled)

35. (canceled)

36. (canceled)

37. (canceled)

38. (canceled)

39. A transducer for ultrasound measuring, the transducer comprising:
a piezoelectric layer configured to resonate around a predetermined ultrasound wavelength and frequency; and
a conductive backing layer directly connected to the bottom side of the piezoelectric layer, the conductive backing layer having a thickness that produces about −20 dB or less of round-trip attenuation, wherein the conductive backing layer is configured to operate as a conductive electrode of the transducer.

40. The transducer of claim 39, further comprising a rigid body over which the conductive backing layer is positioned, the rigid body assembled for encompassing a central longitudinal axis of an acoustic probe body, wherein the rigidity of the rigid body is configured to attenuate ultrasound signals directed toward the central longitudinal axis of the acoustic probe body and to direct ultrasound signals away from the central longitudinal axis of the acoustic probe body.

41. (canceled)

42. (canceled)

43. The transducer of claim 39, further comprising a metallic conductive matching layer positioned over a top side of the piezoelectric layer, wherein the metallic conductive matching layer has a thickness of less than about a quarter of a resonant wavelength of a material of the matching layer.

44. (canceled)

45. (canceled)

46. (canceled)

47. (canceled)

48. An ultrasound probe assembly comprising:
a plurality of transducers integrated with a probe body, each of the plurality of transducers comprising:
a piezoelectric layer configured to resonate around a predetermined ultrasound wavelength and frequency;
a signal and ground electrode, wherein the ground electrodes of each of the transducers comprise a common metallic layer formed over a top of each of the piezoelectric layers of the plurality of transducers; and
a plurality of electrical waveguides extending from a proximal end of the probe body to the plurality of transducers and connected to corresponding signal and ground electrodes of the plurality of transducers.

49. The ultrasound probe assembly of claim 48, wherein the common metallic layer comprises at least one of chrome, gold, platinum, or copper.

50. The ultrasound probe assembly of claim 48, further comprising an electrode positioned below the piezoelectric layer of each of the plurality of transducers, the electrode configured to damp ultrasound waves generated by the piezoelectric layer.

51. (canceled)

52. The ultrasound probe assembly of claim 48, wherein the plurality of electrical waveguides comprises at least one elongated flexible printed circuit.

53. (canceled)

54. (canceled)

55. (canceled)

56. The ultrasound probe assembly of claim 48, wherein the width of the piezoelectric layer is at least about a wavelength of a resonant frequency of an external environment outside of the ultrasound probe assembly.

57.-80. (canceled)

81. An ultrasound measuring probe assembly comprising:
a plurality of transducers integrated with a probe body, each of the plurality of transducers comprising a piezoelectric layer configured to resonate around a predetermined ultrasound wavelength and frequency and wherein the plurality of transducers has a center-to-center pitch of at least about twice the wavelength of the transducers' resonant frequency in an external environment outside of the ultrasound measuring probe assembly; and
a plurality of electrical waveguides extending from a proximal end of the probe body to the plurality of transducers and connected to respective electrodes of the plurality of transducers.

82.-119. (canceled)
